(19) United States
(12) Patent Application Publication
(10) Pub. No.: US 2025/0032636 A1
(43) Pub. Date: Jan. 30, 2025

(54) Phage-Drug Conjugate

(71) Applicants: The University of Sydney, New South Wales (AU); The Westmead Institute for Medical Research, Westmead (AU); Western Sydney Local Health District, Westmead (AU)

(72) Inventors: Thi Thu Hien Duong, New South Wales (AU); Jonathan Iredell, New South Wales (AU); Huiping Huang, New South Wales (AU)

(21) Appl. No.: 18/713,864

(22) PCT Filed: Nov. 29, 2022
(86) PCT No.: PCT/AU2022/051426

§ 371 Date: May 28, 2024

(30) Foreign Application Priority Data
Nov. 29, 2021 (AU) 2021903857

(51) Int. Cl. A61K 47/69; A61K 47/60; A61P 31/04

(52) U.S. Cl. CPC A61K 47/6901; A61K 47/60; A61P 31/04

(57) ABSTRACT

The present invention relates to phage-drug conjugates and methods for preparing phage-drug conjugates. The present invention also relates to methods of using the phage-drug conjugates.

### CROSS-REFERENCE TO RELATED APPLICATIONS

This application is the United States national phase of International Patent Application No. PCT/AU2022/051426 filed Nov. 29, 2022, and claims priority to Australian Patent Application No. 2021903857 filed Nov. 29, 2021, the disclosures of which are hereby incorporated by reference in their entireties.

### BACKGROUND OF THE INVENTION

### Field of the Invention

The present invention relates to phage-drug conjugates. The present invention also relates to methods for preparing phage-drug conjugates. The present invention further relates to methods of using the phage-drug conjugates.

### Description of Related Art

The misuse and overuse of antibiotics has significantly increased the emergence of antimicrobial resistance. Bacteria can develop defences against antimicrobial therapies, rendering these lifesaving drugs ineffective. The problem of antimicrobial resistance is posing a high risk for health and healthcare systems globally. Antimicrobial-resistant pathogens of particular concern include the ESKAPE pathogens—Enterococcus faecium, Staphylococcus aureus, Klebsiella pneumoniae, Acinetobacter baumannii, Pseudomonas aeruginosa and Enterobacter spp.—which are responsible for causing various infections in humans, especially in hospital admitted patients and immunocompromised people. These pathogens can potentially evade the host immune defence system and resist antibiotics. New antibiotics are urgently needed; however, the development of new antibiotics is typically a lengthy and costly process and is quickly undermined by antimicrobial resistance.

Embedding bacterial cells in an extracellular polymeric matrix is the critical adaptive mode for the survival and persistence of bacteria against physical and chemical challenges in their living environment. Many pathogens colonize or develop into a biofilm in a variety of sites in the host body and consequently infect them by degrading the host immune response. Pseudomonas aeruginosa (P. aeruginosa) is a well-known example of a pathogen of concern, causing an estimated 10-20% of all hospital-acquired infections. Along with other biofilm-forming agents, especially the ESKAPE bacteria, this opportunistic pathogen is known to cause acute and chronic infections, such as urinary tract infections, chronic lung infections, and colonise chronic wounds. These infections challenge current treatment regimens due to the difficulty in eradicating persistent biofilms. P. aeruginosa biofilms in the lungs of patients with cystic fibrosis are of particular concern.

Accordingly, there is a need for therapies that can be used for the treatment of bacterial infections, including those that are active in biofilms and those caused by antimicrobial-resistant pathogens.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present inventors have developed phage-drug conjugates which are capable of preventing and/or treating bacterial infections, including those caused by antimicrobial-resistant pathogens.

Accordingly, in one aspect the present invention provides a conjugate of a phage linked with one or more types of active agent by a linker, wherein the phage is conjugated to the linker by one or more types of cleavable bond and the one or more types of active agent are each bound to the linker.

The one or more types of active agent may be directly or indirectly bound to the linker.

In one embodiment, the one or more types of active agent are each independently directly bound to the linker. Preferably, the one or more types of active agent are each independently conjugated to the linker by one or more types of cleavable bond, thereby the one or more types of active agent are each independently directly bound to the linker.

Accordingly, in one embodiment the present invention provides a conjugate of a phage linked with one or more types of active agent by a linker, wherein the phage and the one or more types of active agent are each independently conjugated to the linker by one or more types of cleavable bond.

In another embodiment, the one or more types of active agent are each indirectly bound to the linker. Accordingly, in one embodiment, the present invention provides a conjugate of a phage linked with one or more types of active agent by a linker, wherein the phage is conjugated to the linker by one or more types of cleavable bond and the one or more types of active agent are each indirectly bound to the linker.

In one aspect of this embodiment, the one or more types of active agent may be each encapsulated within a nanostructure comprising the linker, thereby the one or more types of active agent are each indirectly bound to the linker. Preferably, the nanostructure incorporates one or more linkers. More preferably, one or more linkers form a self-assembled nanostructure.

Accordingly, the present invention provides a conjugate of a phage linked with one or more types of active agent by a linker, wherein the phage is conjugated to the linker by one or more types of cleavable bond and the one or more types of active agent are each encapsulated within a nanostructure comprising the linker, wherein the one or more types of active agent are each indirectly bound to the linker.

In some embodiments, at least one of the one or more types of cleavable bond is pH labile. In preferred embodiments, at least one of the one or more types of cleavable bond are labile in acidic conditions, preferably labile at a pH of from about 4.0 to about 6.0, more preferably labile at about pH 5.5.

In preferred embodiments, at least one of the one or more types of cleavable bond is an imine.

In preferred embodiments, the one or more types of cleavable bond are the same. The cleavable bond is preferably an imine.

In some embodiments, the linker comprises a polymer which comprises the one or more types of cleavable bond. The polymer is preferably prepared by reversible addition-fragmentation chain-transfer (RAFT) polymerisation.

In some embodiments, the polymer comprises a diblock copolymer comprising a first block and a second block. In these embodiments, one of the first block and the second block may comprise the one or more types of cleavable bond, preferably the second block.

In preferred embodiments, the first block comprises poly(oligo(ethylene glycol) methyl ether acrylate) (POEGA).

In preferred embodiments, the second block comprises a polymer composed of repeating monomer unit comprising one type of cleavable bond. The monomer unit preferably has the following structure:

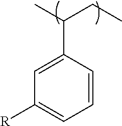

wherein
R is independently —CHO or —C═NZ; and
Z is independently a phage or an active agent.

In preferred embodiments, the conjugate has the following structure:

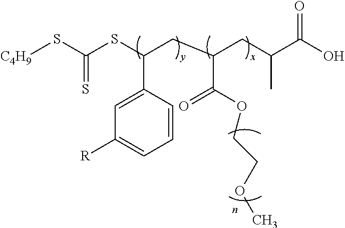

wherein
R is independently —CHO or —C═NZ;
Z is independently a phage or an active agent;
n is 9;
x is 10 to 30, preferably 20 to 28, more preferably 20; and
y is 5 to 15, preferably 10.

In some embodiments, the phage is capable infecting one or more pathogens selected from Escherichia coli, Enterococcus faecium, Staphylococcus aureus, Klebsiella pneumoniae, Acinetobacter baumannii, Pseudomonas aeruginosa and Enterobacter spp. In preferred embodiments, the phage is capable of infecting Escherichia coli or Pseudomonas aeruginosa. In one preferred embodiment, the phage is capable of infecting Escherichia coli. In another preferred embodiment, the phage is capable of infecting Pseudomonas aeruginosa.

In preferred embodiments, the phage is selected from a filamentous phage, an MS2 phage and a caudovirus. In preferred embodiments, the phage is a caudovirus.

In some embodiments, the one or more types of active agent comprise one or both of (i) one or more antibacterial agents and (ii) one or more antibiofilm agents. Preferably the one or more antibiofilm agents comprise a nitroxide group.

In some embodiments, the conjugate comprises, or further comprises, one or more types of imaging agent conjugated to the linker. In embodiments where an imaging agent is also suitable for use in therapy, the imaging agent and the active agent may be the same.

In another aspect the present invention provides a method for preparing a conjugate of a phage linked with one or more types of active agent by a linker, the method comprising:
providing a linker comprising one or more types of reactive functionality capable of forming a cleavable bond;
binding one or more types of active agent to the linker; and
conjugating a phage to the linker via at least one of the one or more types of reactive functionality;
   thereby providing the conjugate, wherein the phage is conjugated to the linker by one or more types of cleavable bond and the one or more types of active agent are each bound to the linker.

In another aspect the present invention provides a method for preparing a conjugate of a phage linked with one or more types of active agent by a linker, the method comprising:
providing a linker comprising one or more types of reactive functionality capable of forming a cleavable bond;
conjugating one or more types of active agent to the linker, each active agent independently being conjugated to the linker via at least one of the one or more types of reactive functionality; and conjugating a phage to the linker via at least one of the one or more types of reactive functionality;
thereby providing the conjugate, wherein the phage and the one or more types of active agent are each independently conjugated to the linker by one or more types of cleavable bond.

In another aspect the present invention provides a method for preparing a conjugate of a phage linked with one or more types of active agent by a linker, the method comprising:
providing a linker comprising one or more types of reactive functionality capable of forming a cleavable bond;
encapsulating one or more types of active agent within a nanostructure comprising the linker, each active agent independently being indirectly bound to the linker; and
conjugating a phage to the linker via at least one of the one or more types of reactive functionality;
   thereby providing the conjugate, wherein the phage is conjugated to the linker by one or more types of cleavable bond and the one or more types of active agent are each indirectly bound to the linker.

In some embodiments, at least one of the one or more types of reactive functionality is capable of forming a cleavable bond that is pH labile. In preferred embodiments, at least one of the one or more types of reactive functionality is capable of forming a cleavable bond that is labile in acidic conditions, preferable labile at a pH of from about 4.0 to about 6.0, more preferably labile at about pH 5.5.

In some embodiments, at least one of the one or more types of reactive functionality is capable of forming an imine.

In preferred embodiments, at least one of the one or more types of reactive functionality is an aldehyde.

In preferred embodiments, the one or more types of reactive functionality are the same. The reactive functionality is preferably an aldehyde.

The method described herein may comprise a step of preparing a linker comprising one or more types of reactive functionality capable of forming a cleavable bond.

In some embodiments, the linker comprises a polymer which comprises the one or more types of reactive functionality. The polymer is preferably prepared by reversible addition-fragmentation chain-transfer (RAFT) polymerisation.

In some embodiments, the polymer comprises a diblock copolymer comprising a first block and a second block. In these embodiments, one of the first block or the second block may comprise the one or more types of reactive functionality capable of forming a cleavable bond, preferably the second block.

In preferred embodiments, the first block comprises poly(oligo(ethylene glycol) methyl ether acrylate) (POEGA).

In preferred embodiments, the second block comprises poly(3-vinylbenzaldehyde) (PVBA).

In preferred embodiments, the diblock copolymer comprises POEGA-block-PVBA (POEGA-b-PVBA).

In preferred embodiments, the linker has the following structure:

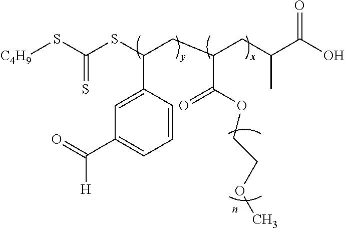

wherein
n is 9;
x is 10 to 30, preferably 20 to 28, more preferably 20; and
y is 5 to 15, preferably 10.

In another aspect, the present invention provides a conjugate prepared by the method described herein.

In another aspect, the present invention provides a pharmaceutical composition comprising the conjugate described herein or prepared by the method of described herein, and a pharmaceutically acceptable diluent, excipient or carrier.

In another aspect, the present invention provides a method for treating a bacterial infection, the method comprising administering the conjugate described herein, the conjugate prepared by the method described herein, or the pharmaceutical composition described herein to an individual in need thereof, thereby treating the bacterial infection.

In another aspect, the present invention provides a method for preventing a bacterial infection, the method comprising administering the conjugate described herein, the conjugate prepared by the method described herein, or the pharmaceutical composition described herein to an individual in need thereof, thereby preventing the bacterial infection.

The individual may be susceptible to developing a bacterial infection. In some embodiments, the individual has cystic fibrosis. In some embodiments, the individual has a prosthetic medical device.

In another aspect, the present invention provides the use of the conjugate described herein or prepared by the method described herein for treating a bacterial infection. The present invention also provides the use of the conjugate described herein or prepared by the method described herein for preventing a bacterial infection.

In another aspect, the present invention provides the use of the conjugate described herein or prepared by the method described herein in the manufacture of a medicament for treating a bacterial infection. The present invention also provides the use of the conjugate described herein or prepared by the method described herein in the manufacture of a medicament for preventing a bacterial infection.

In another aspect, the present invention provides the conjugate described herein or prepared by the method described herein for use in treating a bacterial infection. The present invention also provides the conjugate described herein or prepared by the method described herein for use in preventing a bacterial infection.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. Overlaid ¹H NMR spectra of (a) purified poly(oligo(ethylene glycol) methyl ether acrylate) (POEGA; black line), (b) unpurified (poly(oligo(ethylene glycol methyl ether acrylate)-block-poly(3-vinyl-benzaldehyde) (POEGA-b-PVBA; red line), and (c) purified POEGA-b-PVBA (green line).

FIG. 2. Overlaid ¹H NMR spectra of (a) POEGA-b-PVBA (black line), (b) POEGA-b-PVBA conjugated with 4-TEMPO (green line), (b) POEGA-b-PVBA conjugated with phages (blue line), and (d) POEGA-b-PVBA conjugated with AMK (red line).

FIG. 3. Overlaid ¹H NMR spectra of (a) POEGA-b-PVBA-phage conjugate, (b) POEGA-b-PVBA conjugated with 4-TEMPO, and (c) POEGA-b-PVBA-conjugated with AMK in (i) neutral (black line) or (ii) acidic (pH 5.5; red line) media.

FIG. 4. Graph illustrating molecular weight distributions of polymers POEGA (solid line) and POEGA-b-PVBA (dashed line).

FIG. 5. Overlaid ATR-FTIR spectra of (a) POEGA-b-PVBA (black line), (b) POEGA-b-PVBA conjugated with AMK (red line), (c) POEGA-b-PVBA conjugated with 4-TEMPO (blue line), and (d) POEGA-b-PVBA conjugated with phages (green line). The characteristic aldehyde signal at 1728 cm 1 present in the unconjugated POEGA-b-PVBA is shown in spectrum (a). The characteristic imine signal at 1643 cm 1 present in the POEGA-b-PVBA conjugates is shown in spectra (b)-(d).

FIG. 6. (a) Graphs illustrating effects of antibiotic amikacin (AMK), nitroxide 4-amino-TEMPO, and phages on biofilm dispersal. Bacterial biofilms were grown in multi-well plates for 6 h in the absence of any treatment before being treated with amikacin (4-32 μg/mL), nitroxide (0.25-2 mM) and phages (10²-10⁸ PFU/mL) for 2 h. Biofilm biomass was analysed by crystal violet staining. (b) Graphs illustrating effects of antibiotic, phages and nitroxide on biofilm inhibition. Inoculated cultures were mixed with amikacin (4-32 μg/mL), nitroxide (0.25-2 mM) and phages (10²-10⁸ PFU/mL) for 6 h.

FIG. 7. (a) Graph illustrating effects of different conjugated formulations on biofilm dispersal. Formulations: phages (10⁶ PFU/mL), amikacin (4 μg/mL), amikacin (8 μg/mL), nitroxide (2 mM), phage@nitroxide conjugate, phage@amikacin (4 μg/mL) conjugate, phage@amikacin (8 μg/mL) conjugate, amikacin (4 μg/mL)@nitroxide conjugate, amikacin (8 μg/mL)@nitroxide conjugate, phage@amikacin (4 μg/mL)@nitroxide conjugate, and phage@amikacin (8 μg/mL)@nitroxide conjugate. Bacterial biofilms were grown in multi-well plates for 6 h in the absence of any treatment before being treated with formulations for 2 h. Biofilm biomass was analysed by crystal violet staining. (b) Crystal violet-stained biofilm without treatment and after treatment with nitroxide (2 mM), phages@nitroxide conjugate, phages@AMK (4 μg/mL)@nitroxide conjugate and phages@AMK (8 μg/mL)@nitroxide conjugate. POEGA-b-PVBA linker was used for all conjugates.

FIG. 8. Graphs illustrating effects of different conjugated formulations on P. aeruginosa viability. Formulations: phages (10⁶ PFU/mL), amikacin (4 μg/mL), amikacin (8 μg/mL), nitroxide (2 mM), phage@nitroxide conjugate, phage@amikacin (4 μg/mL) conjugate, phage@amikacin (8 μg/mL) conjugate, amikacin (4 μg/mL)@nitroxide conjugate, amikacin (8 μg/mL)@nitroxide conjugate, phage@amikacin (4 μg/mL)@nitroxide conjugate, and phage@amikacin (8 μg/mL)@nitroxide conjugate. P. aeruginosa biofilms were grown in multi-well plates for 6 h in the absence of any treatments and treated further for 2 h before analysing biofilm cell viability by CFU counting. POEGA-b-PVBA linker was used for all conjugates.

FIG. 9. Representative confocal images showing P. aeruginosa biofilms stained with LIVE/DEAD kit. Biofilms were grown for 6 h and then treated with amikacin, phages, nitroxide, amikacin@nitroxide conjugates and phage@amikacin@nitroxide conjugates for 2 h. Viable and non-viable bacteria appear green and red, as well as those stained both green/red, respectively. Scale bar 10 μm. POEGA-b-PVBA linker was used for all conjugates.

FIG. 10. ¹H NMR spectra of purified POEGA-b-PPFPA. ¹⁹F NMR spectra are given in the inset.

FIG. 11. Graph illustrating molecular weight distributions of polymers POEGA (solid line) and POEGA-b-PPFPA (dashed line).

FIG. 12. ¹⁹H NMR spectra of (a) POEGA-b-PPFPA (t=0), (b) POEGA-b-PPFPA (t=17 h), (c) purified POEGA-b-PPFPA and (d) polymer after the reaction with AMK.

FIG. 13. (a) Effects of different conjugated formulations on biofilm dispersal. Formulations: amikacin (AMK, 8 g/mL), POEGA-b-PPFPA@AMK (8 μg/mL), phages (10⁶ PFU/mL), POEGA-b-PPFPA@phages (10⁶ PFU/mL), nitroxide (2 mM), POEGA-b-PPFP@nitroxide (2 mM), POEGA-b-PPFPA@phages@AMK (8 μg/mL) conjugate, POEGA-b-PPFPA@AMK (8 μg/mL)@nitroxide conjugate, and POEGA-b-PPFPA@phages@AMK (8 μg/mL)@nitroxide conjugate. Bacterial biofilms were grown in multi-well plates for 6 h in the absence of any treatment before being treated with formulations for 2 h. Biofilm biomass was analysed by crystal violet staining. (b) Crystal violet-stained biofilm without treatment and after treatment with nitroxide (2 mM), phages@nitroxide conjugate, phages@AMK (4 μg/mL)@nitroxide conjugate and phages@AMK (8 μg/mL)@nitroxide conjugate. POEGA-b-PPFPA linker was used for all conjugates.

FIG. 14. Effects of different conjugated formulations on P. aeruginosa viability. Formulations: amikacin (AMK, 8 μg/mL), POEGA-b-PPFPA@AMK (8 μg/mL), phages (10⁶ PFU/mL), POEGA-b-PPFPA@phages (10⁶ PFU/mL), nitroxide (2 mM), POEGA-b-PPFPA@nitroxide (2 mM), POEGA-b-PPFPA@phages@AMK (8 μg/mL) conjugate, POEGA-b-PPFPA@AMK (8 μg/mL)@nitroxide conjugate, and POEGA-b-PPFPA@phages@AMK (8 μg/mL)@nitroxide conjugate. P. aeruginosa biofilms were grown in multi-well plates for 6 h in the absence of any treatments and treated further for 2 h before analysing biofilm cell viability by CFU counting. POEGA-b-PPFPA linker was used for all conjugates.

FIG. 15. Overlaid ¹H NMR spectra of (a) POEGA-b-PVBA and (b) phage@CIP nanoparticle conjugates.

FIG. 16. Dynamic light scattering (DLS) analysis of (a) CIP suspension and (b) self-assembled CIP nanoparticles (CIP concentration of 0.1 mg/mL in water). POEGA-b-PVBA linker was used for all conjugates.

FIG. 17 (A) Graphs illustrating effects of phage, CIP nanoparticles, and phage@CIP nanoparticle conjugates on biofilm dispersal. Bacterial biofilms were grown in multi-well plates for 6 h in the absence of any treatment before being treated with CIP nanoparticles (CIP concentration of 5, 10, 20, 40, 80 ng/mL), phages (10⁵ PFU/mL) and phage@CIP nanoparticles (phage titer of 10⁵ PFU/mL and CIP concentration of 5, 10, 20, 40, 80 ng/ml) for 4 h. Biofilm biomass was analysed by crystal violet staining. (B) Graphs illustrating effects of different formulations on E. coli viability. Formulations: CIP nanoparticles (CIP concentration of 5, 10, 20, 40, 80 ng/ml), phages (10⁵ PFU/mL) and phage@CIP nanoparticles (phage titer of 10⁵ PFU/mL and CIP concentration of 5, 10, 20, 40, 80 ng/ml). E. coli biofilms were grown in multi-well plates for 6 h in the absence of any treatments and treated further for 4 h before determining biofilm cell viability by CFU counting. POEGA-b-PVBA linker was used for all conjugates.

FIG. 18 (A) Graphs illustrating effects of phage, CIP nanoparticles, and phage@CIP nanoparticles on biofilm dispersal. Bacterial biofilms were grown in multi-well plates for 6 h in the absence of any treatment before being treated with CIP nanoparticles (CIP concentration of 5, 10, 20, 40, 80 ng/ml), phages (10⁵ PFU/mL) and phage@CIP nanoparticles (phage titer of 10⁵ PFU/mL and CIP concentration of 5, 10, 20, 40, 80 ng/ml) for 24 h. Biofilm biomass was analysed by crystal violet staining. (B) Graphs illustrating effects of different formulations on E. coli viability. Formulations: CIP nanoparticles (CIP concentration of 5, 10, 20, 40, 80 ng/ml), phages (10⁵ PFU/mL) and phage@CIP nanoparticles (phage titer of 10⁵ PFU/mL and CIP concentration of 5, 10, 20, 40, 80 ng/ml). E. coli biofilms were grown in multi-well plates for 6 h in the absence of any treatments and treated further for 24 h before determining biofilm cell viability by CFU counting. POEGA-b-PVBA linker was used for all conjugates.

FIG. 19 Confocal images showing E. coli biofilms stained with LIVE/DEAD kit. Biofilms were grown for 6 h and then treated with CIP nanoparticles (CIP concentration of 20 ng/ml, 2×MIC), phages only (10⁵ PFU/mL) and phage@CIP nanoparticle conjugates (CIP concentration of 20 ng/ml and phage titer of 10⁵ PFU/mL) for 4 h. Viable and non-viable bacteria appear green and red, as well as those stained both green/red, respectively. Scale bar 10 μm. POEGA-b-PVBA linker was used for all conjugates.

### DESCRIPTION OF THE INVENTION

It will be understood that the invention disclosed and defined in this specification extends to all alternative combinations of two or more of the individual features mentioned or evident from the text or drawings. All of these different combinations constitute various alternative aspects of the invention.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

All of the patents and publications referred to herein are incorporated by reference in their entirety.

For the purposes of interpreting this specification, terms used in the singular will also include the plural and vice versa.

Unless defined otherwise, all technical and scientific terms used herein have the same meaning as commonly understood by those of ordinary skill in the art to which the present disclosure belongs. Although any methods and materials similar or equivalent to those described herein can be used in the practice or testing of the present disclosure, preferred methods and materials are described. For the purposes of the present disclosure, the following terms are defined below.

The articles “a” and “an” are used herein to refer to one or to more than one (i.e. to at least one) of the grammatical object of the article. By way of example, “a reactive group” means one reactive group or more than one reactive group.

As used herein, the term “and/or”, e.g., “X and/or Y” will be understood to mean either “X and Y” or “X or Y” and shall be taken to provide explicit support for both meanings or for either meaning.

As used herein, the term “about” refers to a quantity, value, dimension, size, or amount that varies by as much as 10%, 5%, 1% or 0.1% to a reference quantity, value, dimension, size, or amount.

Throughout the present disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

As used herein, unless the context requires otherwise, the term “comprise”, and variations such as “comprises” and “comprising”, will be understood to imply the inclusion of a stated integer or step or group of integers or steps but not the exclusion of any other integer or step or group of integers or steps.

A “subject” herein is preferably a human subject. It will be understood that the terms “subject” and “individual” are interchangeable in relation to an individual requiring administration of the aqueous formulation of the present disclosure.

It will be understood that the invention disclosed and defined in this specification extends to all alternative combinations of two or more of the individual features mentioned or evident from the text. All of these different combinations constitute various alternative aspects of the invention.

The present inventors have developed conjugates comprising a phage and one or more types of active agent conjugated by a cleavable linker. The phage is employed as a drug carrier system for selectively delivering the active agent(s) to a target cell. Advantageously, administering an active agent using a phage-based carrier can allow for controlled, site specific delivery of the active agent, thereby minimising toxic side effects. The conjugate described herein has the potential for wide application by conjugating different active agents and different phages to target different diseases, including a wide variety of bacterial infections. The conjugate has particular application in preventing and/or treating antibiotic resistant infections that require highly toxic, second-line drugs. This is because low amounts of the toxic drug may be administered which, due to the phage site-specific delivery, still achieve a therapeutic local concentration at the site of infection. This has particular application in biofilm infections where common antibiotics lack efficacy. Biofilm infections are associated with prosthetic devices infections as well as infections of bone, heart valves, urinary tract, and particularly in chronic respiratory infections (e.g., cystic fibrosis).

The present invention uses a cleavable linker for conjugating the phage and active agent(s). Irreversible conjugation of the active agent(s) to the phage may be undesirable, for example because the bound phage and/or active agent(s) may be less effective compared to their usual free (unconjugated) form. The use of cleavable (reversible) linker in the conjugate described herein can advantageously allow for controlled release of the active agent(s) from the conjugate at the site of concentration of the phage. As shown in the Examples, conjugates according to the invention were found to effectively disperse biofilms of antimicrobial-resistant pathogen P. aeruginosa or E. coli and reduce the viability of P. aeruginosa or E. coli to a comparable or greater extent than the sole components of the conjugate alone.

### Phage-Drug Conjugate

The present invention provides a conjugate of a phage linked with one or more types of active agent by a linker, wherein the phage is conjugated to the linker by one or more types of cleavable bond and the one or more types of active agent are each bound to the linker. In a preferred embodiment, the phage and the one or more types of active agent are each independently conjugated to the linker by one or more types of cleavable bond. Each of the one or more types of cleavable bond is capable of degrading such that the phage or active agent to which it is conjugated is released upon degradation. In another preferred embodiment, the phage is conjugated to the linker by one or more types of cleavable bond and the one or more types of active agent are each encapsulated within a nanostructure comprising the linker, wherein the one or more types of active agent are each indirectly bound to the linker. Each of the one or more types of cleavable bond is capable of degrading such that the nanostructure is released from the phage upon degradation of the cleavable bond, and the active agent may be released upon disintegration of the dynamic nanostructure. Advantageously, this allows the conjugate to function as a targeted delivery system for therapeutics.

### Cleavable Bond

The conjugate comprises one or more types of cleavable (or degradable) bond. In this context, ‘cleavable bond’ refers to a chemical bond or a chemical group/structure that is able to be cleaved or degraded. The term ‘cleavable bond’ may alternatively be referred to as a ‘cleavable linkage’ or ‘cleavable group’. Each cleavable bond functions to conjugate either a phage or an active agent to the linker, while being capable of degrading such that the phage or active agent to which it is conjugated is released upon degradation.

Each of the one of more types of cleavable bond may be any suitable cleavable bond. The cleavable bond (or cleavable group) may comprise a covalent bond or a non-covalent interaction (e.g. an electrostatic interaction such as an ionic interaction). Suitable cleavable bonds include bonds that are pH labile, i.e., susceptible to degradation in certain pH conditions. In some embodiments, at least one of the one or more types of cleavable bond is labile (or susceptible to degradation) in acidic conditions. Suitable acid-labile cleavable bonds include hydrazones, imines, acetals, ketals, and esters such as boronate esters. Each cleavable bond may be independently labile at a pH of from about 0.5 to about 6.5, for example at a pH of about 0.5, 1.0, 1.5, 2.0, 2.5, 3.0, 3.5, 4.0, 4.5, 5.0, 5.5, 6.0, or 6.5. Any minimum and maximum can be combined to form a range provided that the range is between 0.5 to 6.5, such as a pH of from about 4.0 to about 6.0. In preferred embodiments, at least one of the one or more types of cleavable bond is labile at about pH 5.5.

In some embodiments, at least of the one or more types of cleavable bond is selected from a hydrazone, an imine, an acetal, a ketal, and an ester (e.g., a boronate ester). In preferred embodiments, at least one of the one or more types of cleavable bond is an imine.

The one or more types of cleavable bond present in the conjugate may all be the same. That is, the conjugate may comprise one type of cleavable bond, to which the phage and the one or more types of active agent are each independently conjugated to the linker. In preferred embodiments, the cleavable bond is an imine.

Accordingly, in some aspects, the present invention provides a conjugate of a phage linked with one or more types of active agent by a linker, wherein the phage and the one or more types of active agent are each independently conjugated to the linker by one type of cleavable bond, preferably an imine.

### Linker

The conjugate comprises a linker, to which the phage is conjugated via one or more types of cleavable bond. In some embodiments, the one or more types of active agent are each independently conjugated to the linker by one or more types of cleavable bond. The skilled person will understand that such a linker comprising one or more types of cleavable bond to be a reversible linker. The linker reversibly conjugates either a phage or an active agent to the linker.

The linker may comprise, or consist of, a polymer which comprises the one or more types of cleavable bond. In this context, the term “consist of” means that the linker is entirely composed of the polymer comprising the one or more types of cleavable bond. The polymer may be a homopolymer or a multiblock co-polymer (e.g., a diblock, triblock or tetrablock copolymer). The polymer may comprise any natural polymer, modified natural polymer or synthetic polymer. Suitable natural polymers include polysaccharides such as dextran. Suitable modified natural polymers include modified polysaccharides (e.g., dextran), for example polysaccharides in which hydroxyl groups have been modified to groups capable of forming a cleavable bond (e.g., aldehyde groups). Suitable synthetic polymers include poly(oligo(ethylene glycol) methyl ether acrylate), polyacrylamide, poly(oligo(ethylene glycol) methyl ether methacrylate), poly(3-vinylbenzaldehyde), and modified derivatives thereof.

The polymer may be prepared by any suitable method known in the art. Suitable methods include, but are not limited to, living radical polymerisation techniques, also referred to as controlled radical polymerisation. Controlled radical polymerisation includes but is not limited to: reversible addition/fragmentation chain transfer (RAFT) polymerisation, atom transfer radical polymerisation (ATRP), and nitroxide-mediated polymerisation (NMP). In some embodiments, the polymer is prepared by RAFT polymerisation, for example by the methods described herein. The RAFT agent may be 2-(butylthiocarbonothioylthio) propionic acid (PABTC) or 2-(propylthiocarbonothioylthio)-2-methylpropionoic acid (BPTA), preferably PABTC. In embodiments where the polymer is prepared by RAFT polymerisation, it will be understood that the linker may comprise, or consist of, a polymer having end caps corresponding to the RAFT agent used (e.g., where the RAFT agent is PABTC, a —S—C(═S)—S—C₄H₉ cap and a —C(CH₃)—CO₂H cap).

In some embodiments, the polymer comprises, or consists of, a triblock copolymer comprising a first block, a second block and a third block. In some embodiments, the polymer comprises, or consists of, a diblock copolymer comprising a first block and a second block. In this context, the term “consist of” means that the polymer is entirely composed of the diblock polymer (including end caps, if the polymer is prepared by RAFT polymerisation). In embodiments where the polymer is prepared by RAFT polymerisation, it will be understood that the first block and the second block will each independently comprise an end cap corresponding to the RAFT agent used (e.g., where the RAFT agent is PABTC, either a —S—C(═S)—S—C₄H₉ cap or a —C(CH₃)—CO₂H cap).

The first block and the second block may each be independently composed of a repeating monomer unit (and an end cap, if the polymer is prepared by RAFT polymerisation). The monomer may be any suitable monomer for forming the desired polymer known in the art, including those described herein. In some embodiments, the first block and the second block each independently comprise, or consist of, from 5 to 50 monomer units, for example 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 monomer units. Any minimum and maximum can be combined to form a range provided that the range is between 5 to 50, such as from 5 to 30 monomer units.

One or both of the first block and the second block may comprise the one or more types of cleavable bond, to which the phage and the one or more types of active agent are each independently conjugated. In some embodiments, one of the first block and the second block comprise the one or more types of cleavable bond. In preferred embodiments, the second block comprises the one or more types of cleavable bond.

One or both of the first block and the second block may be useful for tuning one or more properties of the conjugate. For example, one or both of the first and second block may be selected to tune one or more properties of the conjugate relating to its therapeutic use, such as biocompatibility and water solubility. In some embodiments, the first block is selected to tune one or more properties of the conjugate, such as water solubility and biocompatibility. In some embodiments, the first block is hydrophilic. In some embodiments, the first block is biocompatible.

In some embodiments, the first block comprises, or consists of, a polymer composed of 10 to 30 repeating monomer units, for example 10, 11, 12, 13, 14, 15, 16, 17, 18, 19, 20, 21, 22, 23, 24,25, 26, 27, 28, 29 or 30 monomer units. Any minimum and maximum can be combined to form a range provided that the range is between 10 to 30, such as from 20 to 28 monomer units. In this context, the term “consists of” means the first block is entirely composed of the polymer composed of the 10 to 30 repeating monomer units (including end cap, if the polymer is prepared by RAFT polymerisation). In embodiments where the polymer is prepared by RAFT polymerisation, it will be understood that the first block comprises an end cap corresponding to the RAFT agent used (e.g., where the RAFT agent is PABTC, either a —S—C(═S)—S—C₄H₉ cap or a —C(CH₃)—CO₂H cap, preferably a —C(CH₃)—CO₂H cap). In some embodiments, the first block comprises, or consists of, a polymer composed of 20 monomer units.

In some embodiments, the first block comprises, or consists of, a polymer selected from poly(oligo(ethylene glycol) methyl ether acrylate) (POEGA), poly(oligo(ethylene glycol) methyl ether methacrylate) (POEGMA) and polyacrylamide. In some embodiments, the first block comprises, or consists of, POEGA, which is composed of oligo (ethylene glycol) methyl ether acrylate (OEGA) as a repeating monomer unit. The OEGA may have an average Mₙ of 480 g/mol or 2000 g/mol, preferably 480 g/mol. Advantageously, the POEGA may be useful for conferring the conjugate with improved hydrophilicity and/or biocompatibility properties. In some embodiments, the first block comprises, or consists of, POEGMA, which is composed of oligo (ethylene glycol) methyl ether methacrylate (OEGMA) as a repeating monomer unit. The OEGMA may have an average Mₙ of 300 g/mol. In some embodiments, the first block comprises, or consists of, polyacrylamide, which is composed of acrylamide as a repeating monomer unit.

In some embodiments, the first block comprises, or consists of, POEGA which is composed of 10 to 30 OEGA units, for example 10, 11, 12, 13, 14, 15, 16, 17, 18, 19, 20, 21, 22, 23, 24,25, 26, 27, 28, 29 or 30 OEGA units. Any minimum and maximum can be combined to form a range provided that the range is between 10 to 30, such as from 20 to 28 repeating OEGA units. In preferred embodiments, the first block comprises, or consists of, 20 repeating OEGA units.

In some embodiments, the second block comprises, or consists of, a polymer composed of 5 to 15 repeating monomer units, for example 5, 6, 7, 8, 9, 10, 11, 12, 13, 14 or 15 monomer units. Any minimum and maximum can be combined to form a range provided that the range is between 5 and 15, such as a range of 5 to 13 monomer units. In this context, “consist of” means the second block is entirely composed of the repeating monomer unit comprising the one or more types of cleavable bond (including end cap, if the polymer is prepared by RAFT polymerisation). In embodiments where the polymer is prepared by RAFT polymerisation, it will be understood that the second block comprises an end cap corresponding to the RAFT agent used (e.g., where the RAFT agent is PABTC, either a —S—C(═S)—S—C₄H₉ cap or a —C(CH₃)—CO₂H cap, preferably a —S—C(═S)—S—C₄H₉ cap). In some embodiments, the second block comprises, or consists of, 10 repeating monomer units.

The second block may comprise, or consist of, a repeating monomer unit comprising the one or more types of cleavable bond, to which the phage and the one or more types of active agent are each independently conjugated. It will be understood that each monomer unit may independently i) be conjugated to either the phage or one of the one or more types of active agent of the conjugate, or ii) contain an unreacted reactive functionality (e.g., where conjugation efficiency is below 100%). It will be appreciated that, in these embodiments, the second block will comprise at least one conjugated phage and one conjugated active agent.

In some embodiments, the second block comprises, or consists of, a repeating monomer unit comprising one type of cleavable bond. Preferably the cleavable bond is an imine.

In some embodiments, the second block comprises, or consists of, a polymer composed of a repeating monomer unit having the following structure:

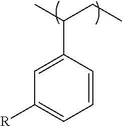

wherein
R is independently —CHO or —C═NZ; and
Z is independently a phage or an active agent.

In some embodiments, the second block comprises, or consist of, a polymer composed of 5 to 15 repeating monomer units having the following structure:

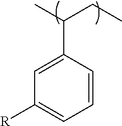

wherein
R is independently —CHO or —C═NZ; and
Z is independently a phage or an active agent,
for example 5, 6, 7, 8, 9, 10, 11, 12, 13, 14 or 15 of the monomer units. Any minimum and maximum can be combined to form a range provided that the range is between 5 to 15, such as a range of 5 to 12 monomer units. In preferred embodiments, the second block comprises, or consists of, 10, of the monomer units.

In preferred embodiments, the conjugate has the following structure:

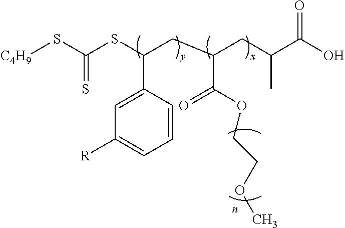

wherein
R is independently —CHO or —C═NZ;
Z is independently a phage or an active agent;
n is 9;
x is 10 to 30, preferably 20 to 28, more preferably 20; and
y is 5 to 15, preferably 10.

It will be appreciated that the conjugate will comprise at least one conjugated phage and one conjugated active agent.

### Phage

The conjugate comprises a phage, which is conjugated to the linker via a cleavable bond. Phages (also called bacteriophages) are viruses that infect and replicate within bacteria and archaea. Phages have been used directly as antibacterial therapies. Phages have also been used as drug carriers or nanobots for targeted drug delivery, due to their drug loading capability as well as their ability to selectively target bacteria and/or to be engineered to selectively target specific cell types. Advantageously, the phage of the conjugate described herein may allow for targeted delivery of the one or more types of active agent to the specific bacteria or cell type targeted by the phage.

The phage of the conjugate described herein may be any phage suitable for use in delivery of an active agent, for example targeted drug delivery, including those known in the art. The phage may be suitably selected depending on the intended target for delivery of the one or more types of active agent. For example, in cases where the intended target is a bacteria (e.g., a pathogenic bacteria), the phage may be capable of targeting that bacteria. As another example, in cases where the intended target is a cancer cell (e.g., a tumour cell), the phage may be capable of targeting that cancer cell. The phage may be genetically engineered to target an intended target. For example, in cases where the intended target is a cancer cell, the phage may be genetically engineered to display one or more ligands on the surface of the phage which are capable of targeting that cancer cell, for example one or more antibodies specific to one or more receptors on the cancer cell. Methods for genetically modifying phages are known in the art.

In some embodiments, the phage is capable of infecting a pathogenic bacteria. Examples of suitable pathogenic bacteria include Escherichia coli, Enterococcus faecium, Staphylococcus aureus, Klebsiella pneumoniae, Acinetobacter baumannii, Pseudomonas aeruginosa and Enterobacter spp. In preferred embodiments, the phage is capable of infecting Pseudomonas aeruginosa. In preferred embodiments, the phage is capable of infecting Escherichia coli.

The phage may be selected from a filamentous phage, an MS2 phage and a caudovirus (e.g. a myovirus such as T4 phage or a siphovirus such as lambda phage). In some embodiments, the phage is a caudovirus, for example a myovirus.

Caudoviruses, such as myoviruses, comprise a head portion and a tail portion (tail fibres). Typically the head portion exhibits an overall net negative charge, and the tail portion exhibits an overall net positive charge. In embodiments of the conjugate described herein where the phage is a caudovirus (e.g. myovirus), preferably the head of the phage is conjugated to the linker. This may advantageously allow the tail portion of the caudovirus to be oriented outwards, which may facilitate target (host) recognition.

### Active Agent

The conjugate comprises one or more types of active agents (or drugs), each of which is bound to the linker.

In one embodiment, the one or more types of active agent are each independently conjugated to the linker by one or more types of cleavable bond. In such embodiments, the one or more types of active agent comprise one or more types of complementary reactive functionality capable of forming a cleavable bond with the one or more types of reactive functionality of the linker.

Preferably, at least one of the one or more types of reactive functionality is capable of forming an imine.

In preferred embodiments, at least one of the one or more types of reactive functionality is an aldehyde.

In preferred embodiments, at least one of the one or more types of complementary reactive functionality is an amine.

The one or more types of active agent may be released from the conjugate by degradation of the cleavable bond.

The one or more types of active agent may be hydrophilic, hydrophobic, or a combination thereof. The one or more types of active agent may comprise none, one or more types of complementary reactive functionality, preferably an amine, capable of forming a cleavable covalent bond with the one or more types of reactive functionality of the linker.

In one embodiment, the one or more types of active agent may be hydrophilic. The one or more types of hydrophilic active agent may comprise one or more types of complementary reactive functionality, preferably an amine, capable of forming a cleavable covalent bond with the one or more types of reactive functionality of the linker. In another embodiment, the one or more types of hydrophilic active agent do not comprise one or more types of complementary reactive functionality. The one or more types of hydrophilic active agent that do not comprise one or more types of complementary reactive functionality may form a cleavable charge interaction bond with the one or more types of reactive functionality of the linker.

In another embodiment, the one or more types of active agent may be hydrophobic. The one or more types of hydrophobic active agent may comprise one or more types of complementary reactive functionality, preferably an amine, capable of forming a cleavable covalent bond with the one or more types of reactive functionality of the linker. In another embodiment, the one or more types of hydrophobic active agent do not comprise one or more types of complementary reactive functionality. The one or more types of hydrophobic active agent that do not comprise one or more types of complementary reactive functionality may be indirectly bound to the linker.

In one embodiment, the one or more types of active agent are each indirectly bound to the linker. In such embodiments, the one or more types of active agent do not comprise one or more types of complementary reactive functionality capable of forming a cleavable bond with the one or more types of reactive functionality of the linker. In such embodiments, preferably the one or more types of active agent do not comprise an amine.

In preferred embodiments, the one or more types of active agent that do not comprise one or more types of complementary reactive functionality, may be each encapsulated within a nanostructure comprising the linker. Preferably, the one or more types of active agent that do not comprise one or more types of complementary reactive functionality form a self-assembled nanostructure with the linker wherein the one or more types of active agent are each indirectly bound to the linker.

The one or more types of active agents may be any suitable active agent. Each active agent may independently be a small molecule drug or a macromolecular drug. In some embodiments, the one or more types of active agent comprise one or more small molecule drugs. In some embodiments, the one or more types of active agent are small molecule drugs. The small molecule drug(s) may be any suitable organic compound having a low molecular weight (less than about 900 daltons) and that can regulate a biological process to treat a particular disease or condition. As shown in the Examples, conjugates described herein are capable of releasing small molecule drugs conjugated to the conjugate by a cleavable bond. In some embodiments, the one or more types of active agent comprise one or more macromolecular drugs. Macromolecular drugs useful in the invention include large molecules (molecular weight more than about 900 daltons) such as proteins, polysaccharides and nucleic acids and that can regulate a biological process to treat a particular disease or condition.

The active agent(s) may be suitably selected, for example, depending on the intended target for delivery of the active agent(s) and/or the intended application of the conjugate. For example, in cases where the intended target may be a bacteria (e.g., a pathogenic bacteria), the one or more types of active agent may comprise one or more antibacterial agents and/or an antibiofilm agents, including those known in the art. As another example, in cases where the intended target may be a cancer cell (e.g., a tumour cell), the one or more types of active agent may comprise one or more anticancer agents, including those known in the art. Advantageously, the capability of the conjugate for targeted delivery may allow for safe administration of otherwise toxic drugs, for example second-line antibiotic drugs. Accordingly, in some embodiments, the one or more types of active agent comprise one or more second-line antibiotic drugs, including those described herein.

In some embodiments, the one or more types of active agent comprise one or more antibacterial agents. Any suitable antibacterial agent may be used, including those known in the art. The one or more antibacterial agents may comprise an antibacterial agent useful for treating a disease and/or an infection caused by an antibiotic-resistant bacteria. The one or more antibacterial agents may comprise a second-line antibacterial agent. Examples of suitable antibacterial agents include aminoglycoside antibiotics such as amikacin, gentamicin, neomycin, kanamycin, neomycin, tobramycin and streptomycin; antimicrobial peptides including octapeptides, migainins, and polymyxins such as polymixin B and polymyxin E (colistin); and fluoroquinolone antibiotics such as ciprofloxacin and gemifloxacin. In some embodiments, the one or more antibacterial agents comprise one or more of amikacin, colistin, gentamicin and ciprofloxacin.

The one or more antibacterial agents may comprise an antibiotic useful for treating an infection caused by a pathogenic bacteria selected from Escherichia coli, Enterococcus faecium, Staphylococcus aureus, Klebsiella pneumoniae, Acinetobacter baumannii, Pseudomonas aeruginosa and Enterobacter spp. In preferred embodiments, the one or more antibacterial agents comprise an antibiotic useful for treating an infection of caused by Pseudomonas aeruginosa. In preferred embodiments, the one or more antibacterial agents comprise an antibiotic useful for treating an infection of caused by Escherichia coli.

In some embodiments, the one or more types of active agent comprise one or more antibiofilm agents. Any suitable antibiofilm agent may be used, including those known in the art. The one or more antibiofilm agents may each be independently capable of one or more of the following: inhibiting (or preventing) biofilm formation, dispersing biofilm, and treating (or eradicating) biofilm. The one or more antibiofilm agents may comprise one or more of the following: one or more biofilm-inhibiting agents, one or more biofilm-dispersing agents and one or more biofilm eradication agents. Examples of suitable antibiofilm agents include nitric oxide (NO) donors and nitroxide-containing antibiofilm agents such as 4-amino-2,2,6,6-tetramethyl-piperidinyloxyl (4-amino TEMPO). In some embodiments, the one or more antibiofilm agents comprise a nitroxide-containing antibiofilm agent, preferably 4-amino TEMPO.

The one or more antibiofilm agents may comprise an antibiofilm agent useful for inhibiting, dispersing and/or treating biofilm formed by a pathogenic bacteria selected from Escherichia coli, Enterococcus faecium, Staphylococcus aureus, Klebsiella pneumoniae, Acinetobacter baumannii, Pseudomonas aeruginosa and Enterobacter spp. In preferred embodiments, the one or more antibiofilm agents comprise an antibiofilm agent useful for inhibiting, dispersing and/or treating biofilm formed by Pseudomonas aeruginosa. In preferred embodiments, the one or more antibiofilm agents comprise an antibiofilm agent useful for inhibiting, dispersing and/or treating biofilm formed by Escherichia coli.

In some embodiments, the one or more types of active agent comprise one or more anticancer agents. Any suitable anticancer agent may be used, including those known in the art. Suitable anticancer agents include chemotherapeutic agents, for example anthracyclines such as doxorubicin, topoisomerase inhibitors such as mitoxantrone, and platinum-containing anticancer agents such as cisplatin and the like; and radiotherapy agents, for example radionuclides suitable for use in radiation therapy. In some embodiments, the one or more anticancer agents comprise a chemotherapeutic agent. In some embodiments, the one or more anticancer agents comprise a radiotherapy agent, preferably a radionuclide suitable for therapy. The radionuclide may additionally be suitable for imaging or diagnostic purposes. Such radionuclides may be suitable for therapeutic applications as well as diagnostic and theranostic applications. Suitable radionuclides include beta emitters, gamma emitters and positron emitters, for example carbon-11 (¹¹C), carbon-14 (¹⁴C), fluorine-18 (¹⁸F), gallium-67 and -68 (⁶⁷Ga, ⁶⁸Ga), yttrium-90 (⁹⁰Y), technetium-99 (^{99m}Tc), indium-111 (¹¹¹In), iodine-123, -125 and -131 (¹²³I, ¹²⁵I, ¹³¹I) and lutetium-177 (¹⁷⁷Lu). In preferred embodiments, the radionuclide is gallium-67. The radionuclide may be directly linked to the cleavable bond or indirectly linked (e.g., via a spacer or chelator moiety that is linked to the cleavable bond). In some embodiments, the radionuclide is indirectly linked to the cleavable bond, for example via a spacer or via a chelator moiety to which the radionuclide is complexed. In these embodiments, the radionuclide may be linked to the spacer or chelator moiety by a covalent bond or a non-covalent bond (e.g., by coordination).

In some embodiments, the one or more types of active agent comprise one or more disinfectant agents.

### Imaging Agent

The conjugate of the present invention may comprise, or may further comprise, one or more types of imaging agent conjugated to the linker.

The one or more types of imaging agent may be any suitable agent that can allow for visualisation of the conjugate via appropriate imaging techniques known in the art. Suitable imaging agents include chromophores, fluorophores and radionuclides. The imaging agent may additionally be suitable for therapeutic purposes. It will therefore be appreciated that in some embodiments the imaging agent and the active agent may be the same, for example the imaging agent may be an anti-cancer agent such as an anthracycline (e.g. doxorubicin) or a radionuclide suitable for therapy. In some embodiments, the one or more imaging agents comprise an anthracycline, for example doxorubicin. In some embodiments, the one or more imaging agents comprise a radionuclide, preferably a radionuclide suitable for imaging or diagnostic purposes. The radionuclide may additionally be suitable for therapy, for example suitable for use as an anticancer agent. Such radionuclides may be suitable for diagnostic application as well as therapeutic and theranostic applications. Suitable radionuclides include beta emitters, gamma emitters and positron emitters. In some embodiments, the radionuclide is selected from carbon-11 (¹¹C), fluorine-18 (¹⁸F), scandium-44 (⁴⁴Sc), copper-62, -64 and -67 (⁶²Cu, ⁶⁴Cu, ⁶⁷Cu), gallium-67 and -68 (⁶⁷Ga, ⁶⁸Ga), yttrium-86 and -90 (⁸⁶Y, ⁹⁰Y), zirconium-89 (⁸⁹Zr), niobium-90 (⁹⁰Nb), technetium-94 and -99 (^{94m}Tc, ^{99m}Tc), indium-111 (¹¹¹In), iodine-123, -124, -125 and -131 (¹²³I, ¹²⁴I, ¹²⁵I, ¹³¹I), lutetium-177 (¹⁷⁷Lu) and bismuth-123 (²¹³Bi). In some embodiments, the radionuclide is selected from a radioisotope of C, F, Sc, Cu, Ga, Ym Zr, Nb, Tc, In, I, Lu and Bi. In preferred embodiments, the radionuclide is gallium-67.

The one or more types of imaging agent may be linked to the conjugate by one or more types of cleavable (reversible) bond, including the one or more types of cleavable bond described herein. In these embodiments, the one or more types of imaging agent may be released from the conjugate by degradation of the cleavable bond. Additionally, or alternatively, the one or more types of imaging agent may be linked to the conjugate by one or more types of non-cleavable (irreversible) bond, for example a bond that is not pH labile. In some embodiments, each of the one or more types of imaging agent are independently linked to the conjugate by a non-cleavable bond, preferably a bond that is not pH labile. The one or more types of imaging agent may be directly linked to the cleavable bond or indirectly linked (e.g., via a spacer or chelator moiety that is linked to the cleavable bond). For example, in embodiments where the one or more types of imaging agent comprise a radionuclide, the radionuclide may be indirectly linked to the cleavable bond, for example via a spacer or via a chelator moiety to which the radionuclide is complexed. In these embodiments, the radionuclide may be linked to the spacer or chelator moiety by a covalent bond or a non-covalent bond (e.g., by coordination).

In embodiments where the linker of the conjugate is a polymer, the one or more types of imaging agent may be linked to the conjugate via a monomer unit of the polymer. In embodiments where the polymer is prepared by RAFT polymerisation, the one or more types of imaging agent may additionally or alternatively be conjugated to an end cap of the polymer corresponding to the RAFT agent used to prepare the polymer. It will be appreciated that in embodiments where the one or more types of imaging agent are linked to the conjugate by a non-cleavable bond, the one or more types of imaging agent may preferably be linked to the polymer via a different portion of the polymer than the phage and the one or more active agents. For example, each of the one or more types of imaging agent may be independently conjugated to a different polymer block (e.g., in embodiments where the polymer is a multiblock polymer, such as a diblock or triblock copolymer) or an end cap of the polymer (e.g., in embodiments where the polymer is prepared by RAFT polymerisation).

### Methods of Preparation

The present invention also provides a method for preparing a conjugate of a phage linked with one or more types of active agent by a linker.

The method comprises a step of providing a linker comprising one or more types of reactive functionality capable of forming a cleavable bond. The method may additionally, or alternatively, comprise a step of preparing the linker.

The one or more types of reactive functionality may comprise a reactive functionality capable of forming a cleavable bond (or degradable bond) that is pH labile. In some embodiments, the one or more types of reactive functionality comprise a reactive functionality capable of forming a cleavable bond that is labile (or susceptible to degradation) in acidic conditions. Suitable acid-labile cleavable bonds include hydrazones, imines, acetals, ketals, and esters such as boronate esters. Suitable reactive functionalities for forming acid-labile cleavable bonds include aldehydes, ketones, hydrazines, amines (e.g., primary amines), alcohols, and carboxylic acids. The cleavable bond may be labile at a pH of from about 0.5 to about 6.5, for example at a pH of about 0.5, 1.0, 1.5, 2.0, 2.5, 3.0, 3.5, 4.0, 4.5, 5.0, 5.5, 6.0, or 6.5. Any minimum and maximum can be combined to form a range provided that the range is between 0.5 to 6.5, such as a pH of from about 4.0 to about 6.0. In preferred embodiments, the cleavable bond is labile at about pH 5.5. In some embodiments, at least one of the one or more types of reactive functionality is capable of forming a cleavable bond selected from a hydrazone, an imine, an acetal, a ketal, and an ester (e.g., a boronate ester). In preferred embodiments, at least one of the one or more types of reactive functionality is capable of forming an imine.

In some embodiments, the one or more types of reactive functionality comprise one or more of an aldehyde, a ketone, a hydrazine, an amine (e.g., a primary amine), an alcohol, and a carboxylic acid. In preferred embodiments, the one or more types of reactive functionality comprise an aldehyde. In these embodiments, the aldehyde may react with an amine of the phage and/or the one or more types of active agent to form an imine. Accordingly, in some embodiments, the phage and/or the one or more types of active agent may each independently comprise an amine, preferably a primary amine.

The one or more types of reactive functionality of the linker may all be the same. That is, the linker may comprise one type of reactive functionality, which subsequently reacts with a complementary reactive functionality of the phage and the one or more types of active agent to form a cleavable bond. In preferred embodiments, the reactive functionality is an aldehyde. In preferred embodiments, the phage comprises an amine, preferably a primary amine. In some preferred embodiments, the one or more types of active agent comprise an amine, preferably a primary amine. In other preferred embodiments, the one or more types of active agent do not comprise an amine, preferably a primary amine.

The linker may comprise, or consist of, a polymer which comprises the one or more types of reactive functionality. In this context, the term “consist of” means that the linker is entirely composed of the polymer comprising the one or more types of reactive functionality. Any polymerisation technique known in the art may be used to form the polymer. The polymerisation technique may, for example, be a radical polymerisation, preferably radical addition fragmentation chain transfer (RAFT) polymerisation. RAFT polymerisation typically involves the polymerisation of a monomer or combination of monomers in solution in the presence of a radical initiator, chain transfer agent (RAFT agent) and heat. Some suitable RAFT conditions are described in the reviews Moad, G.; Rizzardo, E.; Thang, S. H. Accounts of Chemical Research 2008, 41, 1 133 and Moad, G.; Rizzardo, E.; Thang, S. H. Polymer 2008, 49, 1079. The person skilled in the art will be able to determine appropriate conditions, including solvent selection, temperature for reaction and combination of radical initiator and chain transfer agent depending on the monomer or combination of monomers selected.

In some embodiments, the polymer is prepared by RAFT polymerisation in the presence of a RAFT agent. Any suitable RAFT agent may be used. Examples of suitable RAFT agents include thiocarbonylthio compounds such as dithioesters, dithiocarbamates, trithiocarbonates, and xanthates. In some embodiments, the RAFT agent is selected from a dithioester (e.g., dithiobenzoate) and a trithiocarbonate (e.g., 2-(propylthiocarbonothioylthio)-2-methylpropionoic acid (BPTA) or 2-(butylthiocarbonothioylthio) propionic acid (PABTC), preferably PABTC). In embodiments where the polymer is prepared by RAFT polymerisation, it will be understood that the linker may comprise, or consist of, a polymer having end caps corresponding to the RAFT agent used (e.g., where the RAFT agent is PABTC, a —S—C(═S)—S—C₄H₉ cap and a —C(CH₃)—CO₂H cap).

The polymer may comprise, or consist of, a diblock copolymer comprising a first block and a second block. In this context, the term “consist of” means that the polymer is entirely composed of the diblock polymer (including end caps, if the polymer is prepared by RAFT polymerisation). In embodiments where the polymer is prepared by RAFT polymerisation, it will be understood that the first block and the second block will each independently comprise an end cap corresponding to the RAFT agent used (e.g., where the RAFT agent is PABTC, either a —S—C(═S)—S—C₄H₉ cap or a —C(CH₃)—CO₂H cap). The first block and the second block may each be independently composed of a repeating monomer unit (and an end cap, if the polymer is prepared by RAFT polymerisation).

One or both of the first block and the second block may comprise the one or more types of reactive functionality capable of forming a cleavable bond. In some embodiments, one of the first block and the second block comprise the one or more types of reactive functionality. In preferred embodiments, the second block comprises the one or more types of reactive functionality.

In preferred embodiments, the first block comprises, or consists of, poly(oligo(ethylene glycol) methyl ether acrylate) (POEGA). In this context, “consists of” means the first block is entirely composed of the POEGA polymer (including end cap, if the polymer is prepared by RAFT polymerisation). In embodiments where the polymer is prepared by RAFT polymerisation, it will be understood that the first block comprises an end cap corresponding to the RAFT agent used (e.g., where the RAFT agent is PABTC, either a —S—C(═S)—S—C₄H₉ cap or a —C(CH₃)—CO₂H cap).

In preferred embodiments, the first block comprises, or consists of, oligo (ethylene glycol) methyl ether acrylate (OEGA) as a repeating monomer unit. In this context, “consist of” means the first block is entirely composed of repeating OEGA monomer units (including end cap, if the polymer is prepared by RAFT polymerisation). In some embodiments, the first block comprises, or consists of, 10 to 30 repeating OEGA units, for example 10, 11, 12, 13, 14, 15, 16, 17, 18, 19, 20, 21, 22, 23, 24,25, 26, 27, 28, 29 or 30 repeating OEGA units. Any minimum and maximum can be combined to form a range provided that the range is between 10 to 30, such 20 to 28 repeating OEGA units. In preferred embodiments, the first block comprises, or consists of, 20 repeating OEGA units.

The second block may comprise, or consist of, a repeating monomer unit comprising the one or more types of reactive functionality. In this context, “consist of” means the second block is entirely composed of the repeating monomer unit comprising the one or more types of reactive functionality (including end cap, if the polymer is prepared by RAFT polymerisation). In some embodiments, the second block comprises, or consists of 5 to 15 repeating monomer units, for example 5, 6, 7, 8, 9, 10, 11, 12, 13, 14 or 15 repeating monomer units. Any minimum and maximum can be combined to form a range provided that the range is between 20 to 35, such as a range of 25 to 30 monomer units. In preferred embodiments, the second block comprises, or consists of, 28 to 30, more preferably 28, repeating monomer units.

In some embodiments, the second block comprises, or consists of, a repeating monomer unit comprising one type of reactive functionality. Preferably the reactive functionality is an aldehyde.

In preferred embodiments, the second block comprises, or consists of, poly(3-vinylbenzaldehyde) (PVBA). In this context, the term “consists of” means the second block is entirely composed of the PVBA polymer (including end cap, if the polymer is prepared by RAFT polymerisation). In embodiments where the polymer is prepared by RAFT polymerisation, it will be understood that the second block comprises an end cap corresponding to the RAFT agent used (e.g., where the RAFT agent is PABTC, either a —S—C(═S)—S—C₄H₉ cap or a —C(CH₃)—CO₂H cap).

In preferred embodiments, the second block comprises, or consists of, 3-vinylbenzaldehyde (VBA) as a repeating monomer unit. In this context, “consist of” means the second block is entirely composed of repeating VBA monomer units (including end cap, if the polymer is prepared by RAFT polymerisation). In preferred embodiments, the second block comprises, or consists of, 5 to 15, preferably 10, repeating VBA units.

Accordingly, in some embodiments, the linker is a diblock copolymer which is prepared by:
reacting a RAFT agent with a first monomer to provide a first block polymer; and
reacting the first block polymer with a second monomer to provide the diblock copolymer.

Further, in some embodiments, the method further comprises a step of preparing a linker which is a diblock copolymer. The step of preparing the diblock copolymer may comprise:
reacting a RAFT agent with a first monomer to provide a first block polymer; and
reacting the first block polymer with a second monomer to provide the diblock copolymer.

In these embodiments, the RAFT agent may be any suitable RAFT agent known in the art, including those described herein. The first monomer and the second monomer may be any monomer known in the art suitable for use with the conjugate, including the monomers described herein. The percentage conversion of the first and second monomers can be determined by methods known in the art, including those described herein.

In some embodiments, the first monomer is OEGA.

In some embodiments, the first block polymer is a POEGA polymer (including end caps corresponding to the RAFT agent used).

In some embodiments, the POEGA polymer has an average molecular weight of from about 4000 g mol-1 to about 15000 g mol-1, for example an average molecular weight of about 4500 g mol-1, 5000 g mol-1, 5500 g mol-1, 6000 g mol 1, 6500 g mol-1, 7000 g mol-1, 7500 g mol-1, 8000 g mol-1, 8500 g mol-1, 9000 g mol-1, 9500 g mol-1, 10000 g mol 1, 10500 g mol-1, 11000 g mol-1, 11500 g mol-1, 12000 g mol-1, 12500 g mol-1, 13000 g mol-1, 13500 g mol-1, 14000 g mol-1, 14500 g mol-1, or 15000 g mol-1. Any minimum and maximum can be combined to form a range provided that the range is between 4500 g mol-1 to about 15000 g mol 1, such as an average molecular weight of from about 9000 g mol-1 to about 12000 g mol-1. In preferred embodiments, the POEGA polymer has an average molecular weight of about 9000 g mol-1. The molecular weight of the POEGA polymer can be determined by methods known in the art, for example by ¹H NMR spectroscopy analysis and size exclusion chromatography (SEC) as described herein.

The POEGA polymer preferably has a low polydispersity index (PDI). In some embodiments, the POEGA polymer has a PDI of from about 1.05 to about 1.30, for example about 1.05, 1.06, 1.07, 1.08, 1.09, 1.10, 1.11, 1.12, 1.13, 1.14, 1.15, 1.16, 1.17, 1.8, 1.19, 1.20, 1.21, 1.22, 1.23, 1.24, 1.25, 1.26, 1.27, 1.28, 1.29, or 1.30. Any minimum and maximum can be combined to form a range provided that the range is between 1.05 to 1.30, such as a polydispersity of from about 1.05 to 1.25. In preferred embodiments, the POEGA polymer has a polydispersity of about 1.19. The PDI or molecular weight distribution of the POEGA polymer can be determined by methods known in the art, including those described herein, and may be calculated from the following equation: PDI=M_{w}/Mₙ, where M_{w} is the weight-average molecular weight and Mₙ is the number-average molecular weight.

In some embodiments, the second monomer is VBA.

In some embodiments, the diblock copolymer is POEGA-block-PVBA (POEGA-b-PVBA) (including end caps corresponding to the RAFT agent used).

In some embodiments, the POEGA-b-PVBA has an average molecular weight of from about 5000 g mol-1 to about 17000 g mol-1, for example an average molecular weight of about 5000 g mol-1, 5500 g mol-1, 6000 g mol-1, 6500 g mol-1, 7000 g mol-1, 7500 g mol-1, 8000 g mol-1, 8500 g mol-1, 9000 g mol-1, 9500 g mol-1, 10000 g mol-1, 10500 g mol-1, 11000 g mol-1, 11500 g mol-1, 12000 g mol-1, 12500 g mol 1, 13000 g mol-1, 13500 g mol 1, 14000 g mol 1, 14500 g mol 1, 15000 g mol 1, 15500 g mol-1, 16000 g mol-1, 16500 g mol-1, or 17000 g mol-1. Any minimum and maximum can be combined to form a range provided that the range is between 5000 g mol-1 to 17000 g mol-1, such as an average molecular weight of from about 5500 g mol-1 to about 15000 g mol-1. In some embodiments, the POEGA-b-PVBA has an average molecular weight of about 11000 g mol-1. In preferred embodiments, the POEGA-b-PVBA has an average molecular weight of about 15000 g mol-1. The molecular weight of the POEGA-b-PVBA can be determined by methods known in the art, for example by ¹H NMR spectroscopy analysis and size exclusion chromatography (SEC) as described herein.

The POEGA-b-PVBA polymer preferably has a low PDI. In some embodiments, the POEGA-b-PVBA polymer has a PDI of from about 1.05 to about 1.30, for example about 1.05, 1.06, 1.07, 1.08, 1.09, 1.10, 1.11, 1.12, 1.13, 1.14, 1.15, 1.16, 1.17, 1.8, 1.19, 1.20, 1.21, 1.22, 1.23, 1.24, 1.25, 1.26, 1.27, 1.28, 1.29, or 1.30. Any minimum and maximum can be combined to form a range provided that the range is between 1.05 to 1.30, such as a polydispersity of from about 1.05 to 1.25. The PDI of the POEGA-b-PVBA polymer can be determined by methods known in the art including those described herein.

In preferred embodiments, the linker has the following structure:

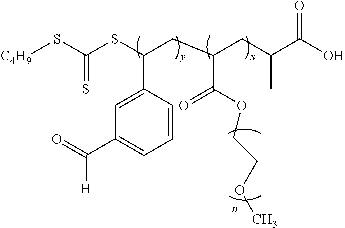

wherein
n is 9;
x is 10 to 30, preferably 20 to 28, more preferably 20; and
y is 5 to 15, preferably 10.

The method of the invention also comprises the steps of:
binding one or more types of active agent to the linker; and
conjugating a phage to the linker via at least one of the one or more types of reactive functionality;
thereby providing the conjugate, wherein the phage is conjugated to the linker by one or more types of cleavable bond and the one or more types of active agent are each bound to the linker.

The method of the invention also comprises the steps of:
conjugating one or more types of active agent to the linker, each active agent independently being conjugated to the linker via at least one of the one or more types of reactive functionality; and
conjugating a phage to the linker via at least one of the one or more types of reactive functionality;
   thereby providing the conjugate, wherein the phage and the one or more types of active agent are each independently conjugated to the linker by one or more types of cleavable bond.

The method of the invention also comprises the steps of:
encapsulating one or more types of active agent within a nanostructure comprising the linker, each active agent independently being indirectly bound to the linker; and
conjugating a phage to the linker via at least one of the one or more types of reactive functionality;
   thereby providing the conjugate, wherein the phage is conjugated to the linker by one or more types of cleavable bond and the one or more types of active agent are each indirectly bound to the linker.

It will be understood that each of the one or more types of reactive functionality of the linker independently react with a respective complementary reactive functionality of the phage or one of the one or more types of active agent, so as to form a cleavable bond which conjugates the phage or active agent respectively to the linker. The complementary reactive functionality of the phage and each of the one or more types of active agent may be the same or different. In preferred embodiments, the complementary reactive functionality of the phage and each of the one or more types of active agent is the same.

The phage may be any phage as described herein. In preferred embodiments, the phage comprises an amine, preferably a primary amine, as a complementary reactive functionality for reacting with a reactive functionality of the linker to form a cleavable bond. In some embodiments, the complementary reactive functionality of the phage is present on the head portion of the phage.

The one or more types of active agents may be any active agent as described herein. The one or more types of active agent may comprise one or more complementary reactive functionality. The complementary reactive functionality of each of the one or more types of active agent may be the same or different. In preferred embodiments, the one or more types of active agent each comprise the same complementary functionality. In preferred embodiments, the one or more types of active agent comprise an amine, preferably a primary amine, as a complementary reactive functionality for reacting with a reactive functionality of the linker to form a cleavable bond.

The one or more types of cleavable bond may be any cleavable bond as described herein. Preferably, the cleavable bond is an imine.

The one or more types of active agent may not comprise one or more complementary reactive functionality.

The steps of conjugating the phage and binding the one or more types of active agent to the linker may be conducted in any order.

Accordingly, in some embodiments, the method comprises:
binding one or more types of active agent to the linker, to provide a conjugate of the linker and the one or more types of active agent; and
conjugating a phage to the conjugate of the linker and the one or more types of active agent via at least one of the one or more types of reactive functionality, to thereby provide the conjugate of the phage linked with the one or more types of active agent by the linker.

Accordingly, in some embodiments, the method comprises:
conjugating one or more types of active agent to the linker, each active agent independently being conjugated to the linker via at least one of the one or more types of reactive functionality, to provide a conjugate of the linker and the one or more types of active agent; and
conjugating a phage to the conjugate of the linker and the one or more types of active agent via at least one of the one or more types of reactive functionality, to thereby provide the conjugate of the phage linked with the one or more types of active agent by the linker.

Accordingly, in some embodiments, the method comprises:
binding one or more types of active agent to the linker, each active agent independently being indirectly bound to the linker, preferably by being encapsulated within a nanostructure comprising the linker, to provide a conjugate of the linker and the one or more types of active agent; and
conjugating a phage to the conjugate of the linker and the one or more types of active agent via at least one of the one or more types of reactive functionality, to thereby provide the conjugate of the phage linked with the one or more types of active agent by the linker.

In other embodiments, the method comprises:
conjugating a phage to the linker via at least one of the one or more types of reactive functionality, to provide a conjugate of the linker and the phage; and
binding one or more types of active agent to the conjugate of the linker and the phage, to thereby provide the conjugate of the phage linked with the one or more types of active agent by the linker.

In other embodiments, the method comprises:
conjugating a phage to the linker via at least one of the one or more types of reactive functionality, to provide a conjugate of the linker and the phage; and
conjugating one or more types of active agent to the conjugate of the linker and the phage, each active agent independently being conjugated to the linker via at least one of the one or more types of reactive functionality, to thereby provide the conjugate of the phage linked with the one or more types of active agent by the linker.

In some embodiments, the step of conjugating the phage is conducted before the step of binding the one or more types of active agent. This may advantageously facilitate conjugation of the phage to the linker by avoiding potential steric hindrance from conjugated active agent(s) limiting conjugation of the phage.

In some embodiments, the step of binding the one or more types of active agents is conducted before the step of conjugating the phage. This may advantageously facilitate the conjugation of a positively-charged conjugate of linker and one or more types of active agent to the negatively charged head portion of a phage.

Any concentration (or amount) of linker, phage and active agent(s) for preparing the conjugate of the invention may be used to achieve a desired level of conjugation of the phage and/or active agent to the linker, and the person skilled in the art will be able to determine appropriate concentrations and conditions. Typically, the concentration of phage and/or active agent reacted with the linker may be selected based on the concentration of the polymer (in particular, the concentration of the reactive functionalities present in the linker).

It will be appreciated that the conjugation efficiency (i.e., the extent to which the reactive functionalities present in the linker react with the phage and the one or more active agents) may vary depending on the amounts of reagents used. The conjugation efficiency may range from i) at least one reactive functionality of the linker has reacted with at least one phage and at least one reactive functionality of the linker has reacted with at least one active agent, to ii) 100% conjugation efficiency (i.e., all reactive functionalities present on the linker have reacted with one or more phages and one or more active agents). For example, the conjugation efficiency may be at least one reactive functionality of the linker has reacted with at least one phage and at least one reactive functionality of the linker has reacted with at least one active agent, about 5%, about 10%, about 15%, about 20%, about 25%, about 30%, about 40%, about 45%, about 50%, about 55%, about 60%, about 65%, about 70%, about 75%, about 80%, about 85%, about 90%, about 95%, or about 100% conjugation efficiency. Any minimum and maximum percentage may be combined to form a range. The conjugation efficiency may also be described herein in terms of the proportion of reactive functionalities present on the linker that have reacted with either i) one or more phages, ii) one or more active agents, or iii) if present, one or more imaging agents. 100% conjugation efficiency may be achieved by using an excess concentration of phage and/or active agent(s) relative to the concentration of the linker (in particular, the concentration of reactive functionalities present in the linker). It will be understood that where the conjugation efficiency is below 100%, the actual percentage conjugation efficiency will depend on the number of reactive functionalities present in the linker. It will also be appreciated that where conjugation efficiency is below 100%, the conjugate produced by the method may comprise one or more unreacted reactive functionalities.

The conjugation of the phage and the one or more types of active agent to the linker can be determined by methods known in the art, for example by ¹H NMR analysis and Attenuated Total Reflection Fourier Transform Infrared (ATR-FTIR) spectroscopy as described herein.

As described herein, the conjugate may further comprise one or more imaging agents conjugate to the linker. Accordingly, in some embodiments, the method further comprises a step of conjugating one or more types of imaging agent to the linker. The one or more types of imaging agent may be any imaging agent as described herein. The step of conjugating the one or more imaging agents may comprise conjugating at least one of the one or more types of imaging agent to the linker via at least one of the one or more types of reactive functionality capable of forming a cleavable (reversible) bond. Additionally, or alternatively, the step may comprise conjugating at least one of the one or more types of imaging agent to the linker via a non-cleavable (irreversible) bond. The one or more types of imaging agent may be conjugated to the linker before, at the same time or after conjugation of one or both of the phage and the one or more active agents to the linker.

Also provided herein is a conjugate prepared by the method described herein.

In preferred embodiments, the conjugate prepared by the method has the following structure:

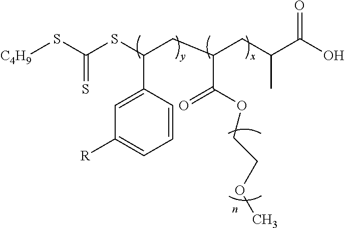

wherein
R is independently —CHO or —C═NZ;
Z is independently a phage or an active agent;
n is 9;
x is 10 to 30, preferably 20 to 28, more preferably 20;
y is 5 to 15, preferably 10.
   As described herein, it will be appreciated that the conjugate will comprise at least one conjugated phage and one conjugated active agent.

### Pharmaceutical Compositions

The present invention also provides a pharmaceutical composition comprising the conjugate described herein or prepared by the method described herein, and a pharmaceutically acceptable diluent, excipient or carrier.

The conjugate or pharmaceutical composition described herein may be administered, or formulated for administration by, any route described herein. As used herein, the term “administered” means administration of a therapeutically effective dose of the compound described herein to the subject. As used herein, the term “formulated for administration” means a therapeutically effective dose of the compound described herein is formulated in such a way that is suitable for the route of administration. In preferred embodiments, the conjugate (or pharmaceutical composition) is administered orally, topically, by nasal administration or parenterally, more preferably parenterally (e.g., intravenously). In other preferred embodiments, the conjugate (or pharmaceutical composition) is formulated for oral administration, topical administration, nasal administration or parenteral administration, more preferably for parenteral administration.

Pharmaceutical compositions may be formulated for any appropriate route of administration including, for example, topical (for example, transdermal or ocular), oral, buccal, nasal, vaginal, rectal or parenteral administration. The term parenteral as used herein includes subcutaneous, intradermal, intravascular (for example, intravenous), intramuscular, spinal, intracranial, intrathecal, intraocular, periocular, intraorbital, intrasynovial and intraperitoneal injection, as well as any similar injection or infusion technique. In certain embodiments, compositions in a form suitable for oral use or parenteral use, especially parenteral use, are preferred. Suitable oral forms include, for example, tablets, troches, lozenges, aqueous or oily suspensions, dispersible powders or granules, emulsions, hard or soft capsules, or syrups or elixirs. Within yet other embodiments, compositions provided herein may be formulated as a lyophilisate.

The various dosage units are each preferably provided as a discrete dosage tablet, capsules, lozenge, dragee, gum, or other type of solid formulation. Capsules may encapsulate a powder, liquid, or gel. The solid formulation may be swallowed, or may be of a suckable or chewable type (either frangible or gum-like). The present invention contemplates dosage unit retaining devices other than blister packs; for example, packages such as bottles, tubes, canisters, packets. The dosage units may further include conventional excipients well-known in pharmaceutical formulation practice, such as binding agents, gellants, fillers, tableting lubricants, disintegrants, surfactants, and colorants; and for suckable or chewable formulations.

Compositions intended for oral use may further comprise one or more components such as sweetening agents, flavouring agents, colouring agents and/or preserving agents in order to provide appealing and palatable preparations. Tablets contain the active ingredient in admixture with physiologically acceptable excipients that are suitable for the manufacture of tablets. Such excipients include, for example, inert diluents such as calcium carbonate, sodium carbonate, lactose, calcium phosphate or sodium phosphate, granulating and disintegrating agents such as corn starch or alginic acid, binding agents such as starch, gelatine or acacia, and lubricating agents such as magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatine capsules wherein the active ingredient is mixed with an inert solid diluent such as calcium carbonate, calcium phosphate or kaolin, or as soft gelatine capsules wherein the active ingredient is mixed with water or an oil medium such as peanut oil, liquid paraffin or olive oil.

Aqueous suspensions contain the active ingredient(s) in admixture with excipients suitable for the manufacture of aqueous suspensions. Such excipients include suspending agents such as sodium carboxymethylcellulose, methylcellulose, hydropropylmethylcellulose, sodium alginate, polyvinylpyrrolidone, gum tragacanth and gum acacia, and dispersing or wetting agents such as naturally-occurring phosphatides (for example, lecithin), condensation products of an alkylene oxide with fatty acids such as polyoxyethylene stearate, condensation products of ethylene oxide with long chain aliphatic alcohols such as heptadecaethyleneoxycetanol, condensation products of ethylene oxide with partial esters derived from fatty acids and a hexitol such as polyoxyethylene sorbitol mono-oleate, or condensation products of ethylene oxide with partial esters derived from fatty acids and hexitol anhydrides such as polyethylene sorbitan monooleate. Aqueous suspensions may also comprise one or more preservatives, for example ethyl, or n-propyl p-hydroxybenzoate, one or more colouring agents, one or more flavouring agents, and one or more sweetening agents, such as sucrose or saccharin.

Oily suspensions may be formulated by suspending the active ingredients in a vegetable oil such as arachis oil, olive oil, sesame oil or coconut oil, or in a mineral oil such as liquid paraffin. The oily suspensions may contain a thickening agent such as beeswax, hard paraffin or cetyl alcohol. Sweetening agents such as those set forth above, and/or flavouring agents may be added to provide palatable oral preparations. Such suspensions may be preserved by the addition of an antioxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, such as sweetening, flavouring and colouring agents, may also be present.

Pharmaceutical compositions may also be in the form of oil-in-water emulsions. The oily phase may be a vegetable oil such as olive oil or arachis oil, a mineral oil such as liquid paraffin, or a mixture thereof. Suitable emulsifying agents include naturally-occurring gums such as gum acacia or gum tragacanth, naturally-occurring phosphatides such as soy bean lecithin, and esters or partial esters derived from fatty acids and hexitol, anhydrides such as sorbitan monoleate, and condensation products of partial esters derived from fatty acids and hexitol with ethylene oxide such as polyoxyethylene sorbitan monoleate. An emulsion may also comprise one or more sweetening and/or flavouring agents.

Syrups and elixirs may be formulated with sweetening agents, such as glycerol, propylene glycol, sorbitol or sucrose. Such formulations may also comprise one or more demulcents, preservatives, flavouring agents and/or colouring agents.

Conjugates described herein may be formulated for local or topical administration, such as for topical application to the skin. Formulations for topical administration typically comprise a topical vehicle combined with active agent(s), with or without additional optional components.

Suitable topical vehicles and additional components are well known in the art, and it will be apparent that the choice of a vehicle will depend on the particular physical form and mode of delivery. Topical vehicles include organic solvents such as alcohols (for example, ethanol, iso-propyl alcohol or glycerine), glycols such as butylene, isoprene or propylene glycol, aliphatic alcohols such as lanolin, mixtures of water and organic solvents and mixtures of organic solvents such as alcohol and glycerine, lipid-based materials such as fatty acids, acylglycerols including oils such as mineral oil, and fats of natural or synthetic origin, phosphoglycerides, sphingolipids and waxes, protein-based materials such as collagen and gelatine, silicone-based materials (both nonvolatile and volatile), and hydrocarbon-based materials such as microsponges and polymer matrices.

A composition may further include one or more components adapted to improve the stability or effectiveness of the applied formulation, such as stabilizing agents, suspending agents, emulsifying agents, viscosity adjusters, gelling agents, preservatives, antioxidants, skin penetration enhancers, moisturizers and sustained release materials. Examples of such components are described in Martindale—The Extra Pharmacopoeia (Pharmaceutical Press, London 1993) and Martin (ed.), Remington's Pharmaceutical Sciences. Formulations may comprise microcapsules, such as hydroxymethylcellulose or gelatine-microcapsules, liposomes, albumin microspheres, microemulsions, nanoparticles or nanocapsules.

A topical formulation may be prepared in a variety of physical forms including, for example, solids, pastes, creams, foams, lotions, gels, powders, aqueous liquids, emulsions, sprays and skin patches. The physical appearance and viscosity of such forms can be governed by the presence and amount of emulsifier(s) and viscosity adjuster(s) present in the formulation. Solids are generally firm and non-pourable and commonly are formulated as bars or sticks, or in particulate form. Solids can be opaque or transparent, and optionally can contain solvents, emulsifiers, moisturizers, emollients, fragrances, dyes/colorants, preservatives and other active ingredients that increase or enhance the efficacy of the final product. Creams and lotions are often similar to one another, differing mainly in their viscosity. Both lotions and creams may be opaque, translucent or clear and often contain emulsifiers, solvents, and viscosity adjusting agents, as well as moisturizers, emollients, fragrances, dyes/colorants, preservatives and other active ingredients that increase or enhance the efficacy of the final product. Gels can be prepared with a range of viscosities, from thick or high viscosity to thin or low viscosity. These formulations, like those of lotions and creams, may also contain solvents, emulsifiers, moisturizers, emollients, fragrances, dyes/colorants, preservatives and other active ingredients that increase or enhance the efficacy of the final product. Liquids are thinner than creams, lotions, or gels, and often do not contain emulsifiers. Liquid topical products often contain solvents, emulsifiers, moisturizers, emollients, fragrances, dyes/colorants, preservatives and other active ingredients that increase or enhance the efficacy of the final product.

Emulsifiers for use in topical formulations include, but are not limited to, ionic emulsifiers, cetearyl alcohol, non-ionic emulsifiers like polyoxyethylene oleyl ether, PEG-40 stearate, ceteareth-12, ceteareth-20, ceteareth-30, ceteareth alcohol, PEG-100 stearate and glyceryl stearate. Suitable viscosity adjusting agents include, but are not limited to, protective colloids or nonionic gums such as hydroxyethylcellulose, xanthan gum, magnesium aluminum silicate, silica, microcrystalline wax, beeswax, paraffin, and cetyl palmitate. A gel composition may be formed by the addition of a gelling agent such as chitosan, methyl cellulose, ethyl cellulose, polyvinyl alcohol, polyquaterniums, hydroxyethylceilulose, hydroxypropylcellulose, hydroxypropylmethylcellulose, carbomer or ammoniated glycyrrhizinate. Suitable surfactants include, but are not limited to, nonionic, amphoteric, ionic and anionic surfactants. For example, one or more of dimethicone copolyol, polysorbate 20, polysorbate 40, polysorbate 60, polysorbate 80, lauramide DEA, cocamide DEA, and cocamide MEA, oleyl betaine, cocamidopropyl phosphatidyl PG-dimonium chloride, and ammonium laureth sulfate may be used within topical formulations.

Preservatives include, but are not limited to, antimicrobials such as methylparaben, propylparaben, sorbic acid, benzoic acid, and formaldehyde, as well as physical stabilizers and antioxidants such as vitamin E, sodium ascorbate/ascorbic acid and propyl gallate. Suitable moisturizers include, but are not limited to, lactic acid and other hydroxy acids and their salts, glycerine, propylene glycol, and butylene glycol. Suitable emollients include lanolin alcohol, lanolin, lanolin derivatives, cholesterol, petrolatum, isostearyl neopentanoate and mineral oils. Suitable fragrances and colours include, but are not limited to, FD&C Red No. 40 and FD&C Yellow No. 5. Other suitable additional ingredients that may be included in a topical formulation include, but are not limited to, abrasives, absorbents, anticaking agents, antifoaming agents, antistatic agents, astringents (such as witch hazel), alcohol and herbal extracts such as chamomile extract, binders/excipients, buffering agents, chelating agents, film forming agents, conditioning agents, propellants, opacifying agents, pH adjusters and protectants.

Typical modes of delivery for topical compositions include application using the fingers, application using a physical applicator such as a cloth, tissue, swab, stick or brush, spraying including mist, aerosol or foam spraying, dropper application, sprinkling, soaking, and rinsing. Controlled release vehicles can also be used, and compositions may be formulated for transdermal administration (for example, as a transdermal patch).

A pharmaceutical composition may be formulated as inhaled formulations, including sprays, mists, or aerosols. This may be particularly preferred for treatment of pulmonary fibrosis. For inhalation formulations, the composition or combination provided herein may be delivered via any inhalation methods known to a person skilled in the art. Such inhalation methods and devices include, but are not limited to, metered dose inhalers with propellants such as CFC or HFA or propellants that are physiologically and environmentally acceptable. Other suitable devices are breath operated inhalers, multidose dry powder inhalers and aerosol nebulizers. Aerosol formulations for use in the subject method typically include propellants, surfactants and co-solvents and may be filled into conventional aerosol containers that are closed by a suitable metering valve.

Inhalant compositions may comprise liquid or powdered compositions containing the active ingredient that are suitable for nebulization and intrabronchial use, or aerosol compositions administered via an aerosol unit dispensing metered doses. Suitable liquid compositions comprise the active ingredient in an aqueous, pharmaceutically acceptable inhalant solvent such as isotonic saline or bacteriostatic water. The solutions are administered by means of a pump or squeeze-actuated nebulized spray dispenser, or by any other conventional means for causing or enabling the requisite dosage amount of the liquid composition to be inhaled into the patient's lungs. Suitable formulations, wherein the carrier is a liquid, for administration, as for example, a nasal spray or as nasal drops, include aqueous or oily solutions of the active ingredient.

Pharmaceutical compositions may also be prepared in the form of suppositories such as for rectal administration. Such compositions can be prepared by mixing the drug with a suitable non-irritating excipient that is solid at ordinary temperatures but liquid at the rectal temperature and will therefore melt in the rectum to release the drug. Suitable excipients include, for example, cocoa butter and polyethylene glycols.

Pharmaceutical compositions may be formulated as sustained release formulations such as a capsule that creates a slow release of modulator following administration. Such formulations may generally be prepared using well-known technology and administered by, for example, oral, rectal or subcutaneous implantation, or by implantation at the desired target site. Carriers for use within such formulations are biocompatible, and may also be biodegradable. Preferably, the formulation provides a relatively constant level of modulator release. The amount of modulator contained within a sustained release formulation depends upon, for example, the site of implantation, the rate and expected duration of release and the nature of the condition to be treated or prevented.

It will be understood that the specific dose level for any particular patient will depend upon a variety of factors including the activity of the specific compound employed, the age, body weight, general health, sex, diet, time of administration, route of administration, and rate of excretion, drug combination (i.e. other drugs being used to treat the patient), and the severity of the particular disorder undergoing therapy.

### Applications

The conjugate described herein may be useful as a drug delivery platform or nanobot for targeted drug delivery. It will be appreciated that the conjugate may be suitable for use in preventing and/or treating any disease or condition for which phage therapy is used as a treatment. The application or intended use of the conjugate may be tailored based on the phage and/or the one or more types of active agent selected for use in the conjugate. For example, in cases where the phage and/or active agent(s) may be selected to target a bacteria (e.g., a pathogenic bacteria), the conjugate may be useful for preventing or treating an infection by that bacteria. As another example, in cases where the phage and/or active agent(s) may be selected to target a cancer cell (e.g., a tumour cell), the conjugate may be useful for preventing or treating that cancer.

As used herein, the terms “treatment” or “treating” of a subject include the application or administration of a conjugate (or pharmaceutical composition) described herein to a subject with the purpose of delaying, slowing, stabilizing, curing, healing, alleviating, relieving, altering, remedying, less worsening, ameliorating, improving, or affecting the disease or condition, the symptom of the disease or condition, or the risk of (or susceptibility to) the disease or condition. The term “treating” refers to any indication of success in the treatment or amelioration of an injury, pathology or condition, including any objective or subjective parameter such as abatement; remission; lessening of the rate of worsening; lessening severity of the disease; stabilization, diminishing of symptoms or making the injury, pathology or condition more tolerable to the subject; slowing in the rate of degeneration or decline; making the final point of degeneration less debilitating; or improving a subject's physical or mental well-being.

As used herein, the term “prevention” or “preventing” are intended to refer to at least the reduction of likelihood of the risk of (or susceptibility to) acquiring a disease or disorder (i.e., causing at least one of the clinical symptoms of the disease not to develop in a patient that may be exposed to or predisposed to the disease but does not yet experience or display symptoms of the disease). Biological and physiological parameters for identifying such patients are provided herein and are known in the art.

Accordingly, the present invention provides a method for preventing or treating a bacterial infection (or a disease associated with a bacterial infection), the method comprising administering the conjugate described herein, the conjugate prepared by the method described herein, or the pharmaceutical composition described herein to an individual in need thereof, thereby preventing or treating the bacterial infection.

The individual may be susceptible to, or at risk of, developing a bacterial infection. For example, the individual may have an existing disease or condition that makes then more susceptible to, or at risk of, developing a bacterial infection. In some embodiments, the individual has cystic fibrosis. In some embodiments, the individual has a prosthetic medical device.

The present invention also provides the use of the conjugate described herein or prepared by the method described herein for preventing or treating a bacterial infection.

The present invention also provides the use of the conjugate described herein or prepared by the method described herein in the manufacture of a medicament for preventing or treating a bacterial infection.

The present invention also provides the conjugate described herein or prepared by the method described herein for use in preventing or treating a bacterial infection.

In these embodiments, the phage of the conjugate is preferably capable of targeting a bacteria (e.g., a pathogenic bacteria) as described herein, and the one or more types of active agent preferably comprise one or both of an antibacterial agent and an antibiofilm agent as described herein.

The bacterial infection may be caused by a pathogenic bacteria, including an antibiotic resistant pathogenic bacteria. In some embodiments, the bacterial infection is caused by a pathogenic bacteria selected from Escherichia coli, Enterococcus faecium, Staphylococcus aureus, Klebsiella pneumoniae, Acinetobacter baumannii, Pseudomonas aeruginosa and Enterobacter spp, preferably Escherichia coli and Pseudomonas aeruginosa.

The present invention also provides a method for preventing or treating a cancer, the method comprising administering the conjugate described herein, the conjugate prepared by the method described herein, or the pharmaceutical composition described herein to an individual in need thereof, thereby preventing or treating the cancer. In these embodiments, the phage of the conjugate is preferably capable of targeting a cancer cell (e.g., a tumour cell) as described herein, and the one or more active agents preferably comprise an anti-cancer agent as described herein. Advantageously, the conjugate described herein may exhibit minimal cytotoxicity to mammalian cells such as human cells. In some embodiments, the cancer is a tumour.

The present invention also provides the use of the conjugate described herein or prepared by the method described herein for preventing or treating a cancer, such as a tumour.

The present invention also provides the use of the conjugate described herein or prepared by the method described herein in the manufacture of a medicament for preventing or treating a cancer, such as a tumour.

The present invention also provides the conjugate described herein or prepared by the method described herein for use in preventing or treating a cancer, such as a tumour.

The conjugate may be provided in any form suitable for its intended application, including forms described herein. The conjugate may be suitable for use in oral, inhalable, parenteral and wound dressing products.

The conjugate described herein is not restricted only to therapeutic uses but may also be useful for industrial applications requiring an antibiotic and/or antibiofilm agent, for example in clearing clogged pipelines and as a food-grade phage spray in commercial kitchens.

As shown in the Examples, the conjugate described herein may advantageously exhibit a comparable or greater therapeutic effect than administration of the individual conjugate components (i.e., the one or more types of active agent and optionally the phage, depending on the intended application) alone. Accordingly, in some embodiments, the therapeutic effect following administration of the conjugate is comparable to or greater than that achieved by administration of the individual components of the conjugate alone. It will be understood that this is based on the conjugate and the individual conjugate components being administered in the same relative amounts and by the same mode of administration. It will be appreciated that even if the conjugate exhibits a similar effect to the individual conjugate components alone, an advantage of using conjugates is the use of the phage of the conjugate as a means to deliver the active agent(s) to targeted sites while avoiding off-site toxicity.

As described herein, the conjugate may comprise (or further comprise) one or more types of imaging agent conjugated to the linker. Advantageously, due to the phage site-specific delivery, an imaging agent may allow for the site of concentration of the phage to be identified. In addition, the detection of the imaging agent may advantageously allow for the identification of whether an individual has a certain disease, disorder or infection, depending on the intended target of the phage. Further, the site-specific delivery of both one or more active agents and one or more imaging agents (where the active agent(s) and imaging agent(s) may be the same or different) may advantageously make them suitable for application in the field of theranostics (i.e., suitable for diagnosis as well as therapy).

Accordingly, the present invention also provides a method for detecting a cell or biological target associated with a disease, disorder or infection in a subject, comprising:
administering to a subject the conjugate described herein or prepared by the method described herein, the conjugate comprising one or more types of imaging agent; and
detecting the conjugate in the subject, wherein presence of the conjugate indicates the presence of the cell or biological target.

The present invention also provides an in vivo method of diagnosing, monitoring or prognosing a disease, disorder or infection in a subject, comprising:
administering to a subject the conjugate described herein or prepared by the method described herein, the conjugate comprising one or more types of imaging agent, and the conjugate specifically localising at a cell or biological target associated with a disease, disorder or infection;
allowing the conjugate to concentrate at sites in the subject where the cell or biological target is found; and
detecting the conjugate;
   whereby detection of the conjugate above a background or standard level indicates that the subject has the disease, disorder or infection.

The present invention also provides a theranostic method for in vitro and/or in vivo visualisation, identification and/or detection of a cell or biological target associated with a disease, disorder or infection as well as a method for treating that disease, disorder or infection. In one embodiment, the present invention includes a theranostic method that comprises administering the conjugate described herein or prepared by the method described herein, the conjugate comprising one or more types of imaging agent, to an individual in need thereof.

As described herein, an active agent may additionally be suitable for diagnostic or imaging purposes, and an imaging agent may be additionally useful for therapeutic purposes. Accordingly, in some methods of the invention, it will be understood that in some embodiments an active agent (e.g., an anticancer agent) and an imaging agent may be the same, for example the conjugate may comprise a radionuclide suitable for therapy as well as imaging or diagnosis.

### Examples

The invention will be further described by way of non-limiting example(s). It will be understood to persons skilled in the art of the invention that many modifications may be made without departing from the spirit and scope of the invention.

### Materials and Methods

### Materials

All antibiotics and chemicals were obtained from Sigma-Aldrich unless otherwise specified. Cation Adjusted Mueller-Hinton (MH) broth was purchased from BD. Luria Bertani (LB) Broth and agar were purchased from Oxoid. P. aeruginosa W.T. PAO1 (wild type PAO1), P. aeruginosa bacteriophage (myovirus) was provided with a titer of 109 PFU mL⁻¹ from the Westmead Institute for Medical Research.

### Phage Propagation and Purification

Propagation of phages was carried out in liquid P. aeruginosa PAO1 culture. P. aeruginosa bacteriophage (myovirus) was added to an exponential phase shaking PAO1 culture at 10⁶-107 CFU/mL and incubate at 37° C. until the culture visibly cleared. The cultures were then centrifuged at 4000 rpm for 20 min then the supernatant was passed through a 0.45-μm pore size filter. The phage concentration was quantified using plaque assay presented as plaque-forming unit per millilitre (PFU/mL).

### (A) Cleavable Linker-Covalent Conjugation

### Synthesis of POEGA Macro-RAFT Agent

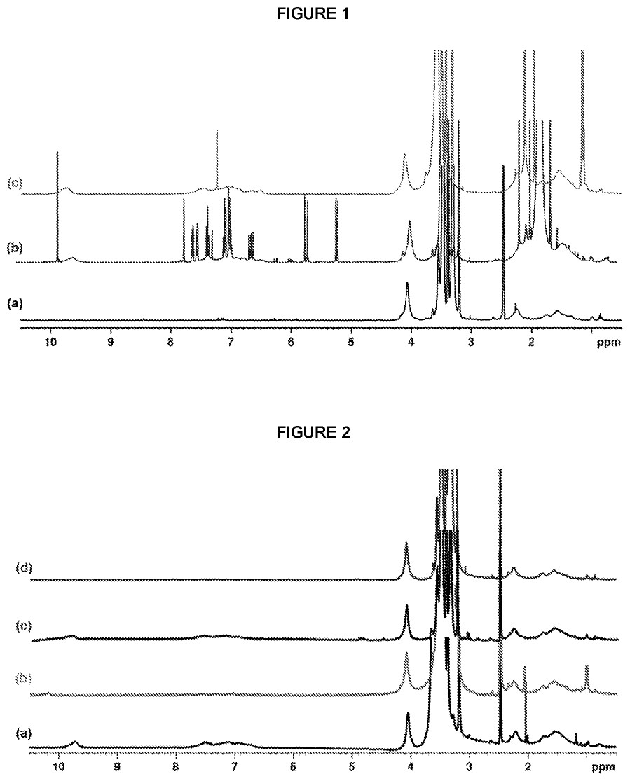

POEGA macro-RAFT agent with 30 repeating units of monomer oligo (ethylene glycol) methyl ether acrylate (OEGA) was synthesised. OEGA (average Mₙ 480 g/mol, 5 g, 1.04×10⁻² mol), 2-(butylthiocarbonothioylthio) propionic acid (PABTC) (8.3×10 2 g, 3.5×10⁻⁴ mol) and 2,2′-azobisisobutyronitrile (AIBN) (5.7×10⁻³ g, 3.5×10 5 mol) were dissolved in 15 mL of toluene in a glass vial and mixed with a magnetic stirrer bar. The vial was sealed with a rubber septum and placed inside an ice bath to halt the polymerisation reaction. The mix was purged with nitrogen gas for 30 min to remove oxygen. The vial was then transferred to a pre-heated oil bath at 70° C. for 17 h. The vial was then placed inside an ice bath for polymerisation termination. The percentage conversion of the OEGA monomer to POEGA was determined via ¹H NMR using deuterated dimethyl sulfoxide (DMSO-d⁶). After confirmation, the POEGA polymer was purified three times by precipitation in excess petroleum spirit, followed by centrifugation at 6000 rpm for 15 min. The purified polymer was dried under vacuum at room temperature and then stored at 4° C. until further use. The ¹H NMR spectrum of the POEGA polymer is shown in FIG. 1(a).

### Synthesis of POEGA-b-PVBA

Chain extension was achieved via introducing the monomer 3-vinyl-benzaldehyde (VBA) with the POEGA macro-RAFT agent. 10-15 repeating units of VBA were conjugated onto the polymer. The synthesised POEGA polymer (2 g, 1.5×10⁻⁴ mol), VBA (3.8×10⁻¹ g, 2.9×10⁻³ mol) and AIBN (6.8×10³ g, 2.9×10⁻⁵ mol) were dissolved in 5 mL of acetonitrile in a round bottom flask. The reaction was halted by placing the flask in an ice bath. The mixture was again purged with nitrogen gas for 30 min to remove oxygen. Next, the flask was heated in a pre-heated oil bath at 70° C. for 17 h. After overnight, the reaction was terminated by placing the flask in an ice bath. The monomer conversion of the VBA monomer to POEGA-b-PVBA polymer was determined via ¹H NMR using deuterated chloroform (CDCl₃). ¹H NMR spectra of the unpurified and purified POEGA-b-PVBA polymer are shown in FIGS. 1(b) & 1(c).

### Conjugation of Active Agents and Phage to POEGA-b-PVBA

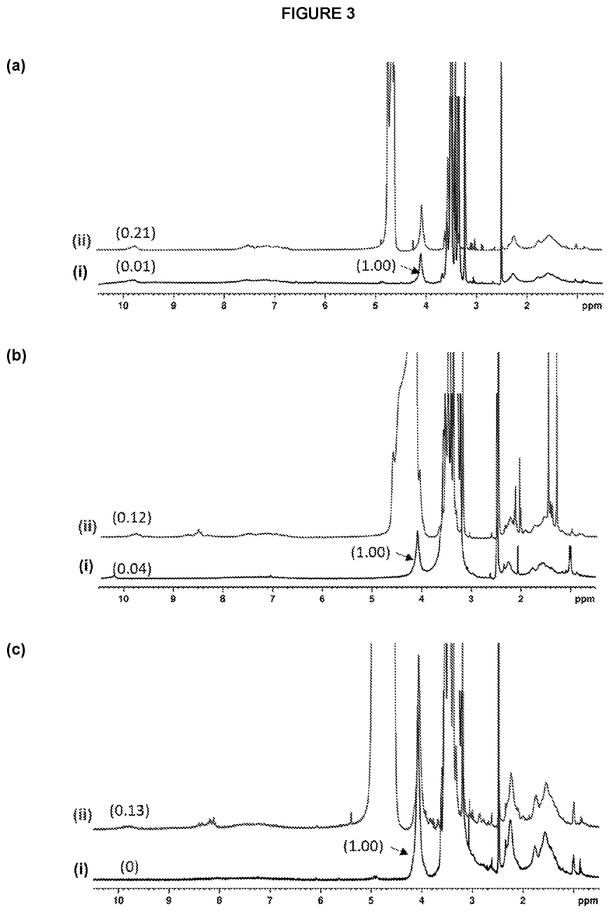

Stock solutions of copolymer POEGA-b-PVBA (10 mg mL⁻¹), amikacin (AMK; 1 mg mL⁻¹) and 4-amino-TEMPO (nitroxide; 40 mM) were separately prepared by dissolving chemicals in Milli Q water and were stored at 4° C. for further use. Various treatment conditions were prepared and incubated at 37° C. with shaking at 180 rpm overnight before adding phages for further conjugation. The attachment of phage on the POEGA-b-PVBA was prepared with shaking at 180 rpm for 6 h before treatment application. The various treatment conditions are shown in Table 1. The attachment and release of individual agents were determined by ¹H NMR. ¹H NMR spectra of the purified POEGA-b-PVBA linker, POEGA-b-PVBA conjugated with nitroxides, POEGA-b-PVBA conjugated with phages and POEGA-b-PVBA conjugated with AMK are shown in FIGS. 2(a)-2(d) respectively. ¹H NMR spectra of the purified POEGA-b-PVBA linker, POEGA-b-PVBA conjugated with phages, and the POEGA-b-PVBA-phage conjugate in neutral and acidic media (pH 5.5) are shown in FIGS. 3(a)-3(c).

TABLE 1
Preparation of POEGA-b-PVBA-phage conjugates

| | V_{polymer} | V_{AMK} | V_{nitroxide} | V_{phage suspension} | V media |
|---|---|---|---|---|---|
| Treatment (1 mL) | (ml) | (ml) | (ml) | (10⁹ PFU/ml) | (ml) |
| Phage-nitroxide | 0.4 | — | 0.05 | 0.02 | 0.53 |
| conjugate | | | | | |
| Phage-AMK(4 μg/mL) | 0.4 | 0.004 | — | 0.02 | 0.576 |
| conjugate | | | | | |
| Phage-AMK(8 μg/mL) | 0.4 | 0.008 | — | 0.02 | 0.572 |
| conjugate | | | | | |
| Phage-AMK(4 μg/mL)- | 0.4 | 0.004 | 0.05 | 0.02 | 0.526 |
| nitroxide conjugate | | | | | |
| Phage-AMK(8 μg/mL)- | 0.4 | 0.008 | 0.05 | 0.02 | 0.522 |
| nitroxide conjugate | | | | | |

### ¹H NMR Spectroscopy Analysis

The percentage conversion of OEGA monomer and polymer composition were characterised by ¹H NMR using Varian 400-MR, Varian (400 MHZ), in DMSO-de solvent. The percentage conversion of OEGA was calculated using equation (1) as follows:

$${α}^{OEGA} = 1 - ( \int 6.3 {ppm}_{( 17 h )} / ( \int 4.2 {ppm}_{( 17 h )} / 2 ) ) \times 100 \qquad ( 1 )$$

The molecular weight of the POEGA polymer was calculated using equation (2), in which M_{W, OEGA} and M_{W·PABTC}=480 g mol⁻¹ and 238 g mol⁻¹, respectively.

$${M}_{n , POEGA} = {α}^{OEGA} \times ( [ OEGA ] / [ PABTC ] ) \times {M}_{W , OEGA} + {M}_{W , PABTC} \qquad ( 2 )$$

For the percentage conversion of VBA, equation (3) was used, as shown below. The percentage conversion of VBA monomer and polymer composition were characterised by ¹H NMR using deuterated CDCl₃ as solvent.

$${α}^{VBA} = 1 - ( \int 9.8 {ppm}_{( 17 h )} / ( \int 9.8 {ppm}_{( 0 h )} ) ) \times 100 \% \qquad ( 3 )$$

The molecular weight of POEGA-b-PVBA was calculated using equation (4), where M_{W, VBA}=132 g mol-1.

$${M}_{n , POEGA - b - PVBA} = {α}^{VBA} \times ( [ VBA ] / [ POEGA ] ) \times {M}_{W , VBA} + {M}_{W , POEGA} \qquad ( 4 )$$

### Size-Exclusion Chromatography (SEC)

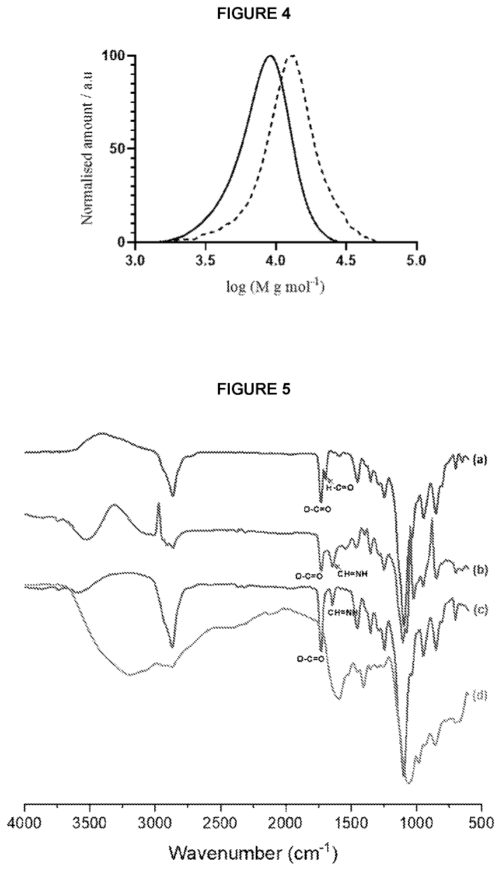

The average molecular weight of POEGA and POEGA-b-PVBA were also determined by size-exclusion chromatography (SEC). The measurements were performed on a UFLC Shimadzu Prominence SEC system with Phenogel™ columns (5 μm, 10⁴, and 10⁵ Å) and a Shimadzu Shim-pack GPC-80DP guard column in N,N-dimethylacetamide (DMAc) with 2,6-di-butyl-4-methylphenol (BHT) and LiBr at 0.05 wt % as eluents at a flow rate of 1 mL min-1 at 50° C. Samples were dissolved and passed through a 220 nm nylon filter before injection. Apparent molecular weights were determined using a calibration procedure with near-monodisperse PMMA standards from Polymer Standards Service (PSS). A graph illustrating the molecular weight distributions of POEGA and POEGA-b-PVBA synthesised as described herein are shown in FIG. 4.

### Antimicrobial Assays

The minimum inhibitory concentration (MIC) of the antimicrobial agents was measured using the broth microdilution technique. A bacterial culture medium with 10⁵-10⁶ CFU mL⁻¹ was prepared freshly in Mueller-Hinton (MH) broth and added to a 2-fold serial dilution of antimicrobial agents in 96-well microplates. A cell-only control and a medium-only control were also included in the same microplate. The 96-well microplates were incubated at 37° C. for 24 h. MIC was determined to be the concentration of antimicrobial agents at which the optical density (OD₆₀₀) was equal to that of a medium-only control using a multimode microplate reader (VICTOR™ X4, Perkin-Elmer). The MIC can also be recorded as the lowest concentration preventing visible bacterial growth after 24 h. The MIC value of AMK and nitroxide were found to be 4 μg mL⁻¹ and 6 mM, respectively, which was in agreement with reported values.

### Biofilm Formation Inhibition and Dispersal Assays

A Pseudomonas aeruginosa PAO1 inoculum was freshly prepared by inoculating one colony in 10 mL of LB broth and incubating on an orbital shaker at 220 rpm at 37° C. for 16-17 h. The bacterium culture medium was diluted 200-fold to an optical density at 600 nm (OD₆₀₀) between 0.003-0.005 in M9 minimal medium (containing 48 mM Na₂HPO₄, 22 mM KH₂PO₄, 9 mM NaCl, 19 mM NH₄Cl, 2 mM MgSO₄, 0.1 mM CaCl₂), and 20 mM glucose, pH 7.0). This medium was freshly prepared for each experiment.

For the biofilm inhibition assay, various treatment formulations were added to the 24-well microtiter plates simultaneously as cell culture media placement. The plates were incubated on an orbital shaker at 180 rpm for 6 h incubation at 37° C. For this 6 h biofilm dispersal assay, the plates were incubated on an orbital shaker at 180 rpm for 6 h at 37° C. for biofilm formation. Planktonic bacteria were removed, and 1 mL of fresh M9 minimal medium was added to each well, followed by each treatment addition. The plates were incubated for a further 2.5-3 h before quantifying the biofilm biomass and viabilities of biofilm bacteria.

The concentrations of each component used for the treatments would be based on their individual effects on biofilm dispersal. Therefore, a dose-response study was conducted based on different minimum inhibitory concentrations (MIC) of AMK (4 and 8 μg mL⁻¹=1× and 2×MIC respectively) while phage titer was 10⁶ PFU mL⁻¹ and nitroxide concentration was 2 mM in the combined therapies.

The biofilm biomass was analysed by crystal violet (CV) staining. The biofilm on each well surface of the plates was washed once with 1 mL PBS before CV staining. 1 mL of 0.1% CV solution made by a 1:20 dilution of 2% CV aqueous solution in PBS. The CV stained biofilm was washed twice with 1 mL PBS after 20 min incubation. The remaining CV stained biofilm was dissolved in 33% glacial acetic acid and quantified by measuring the absorbance at 550 nm (OD₅₅₀) using a multimode microplate reader (VICTOR™ X4, Perkin-Elmer). All biofilm assays were performed in two independent experiments.

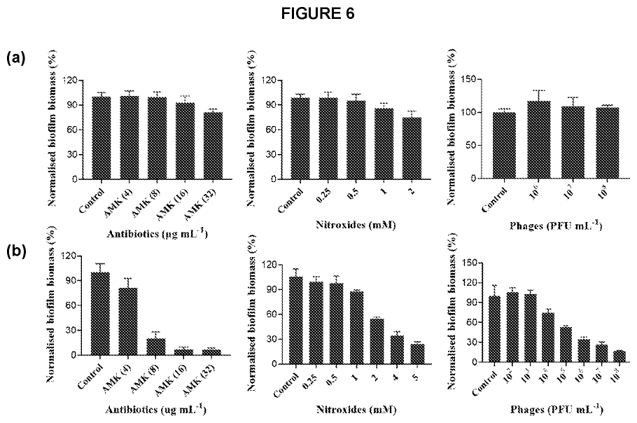

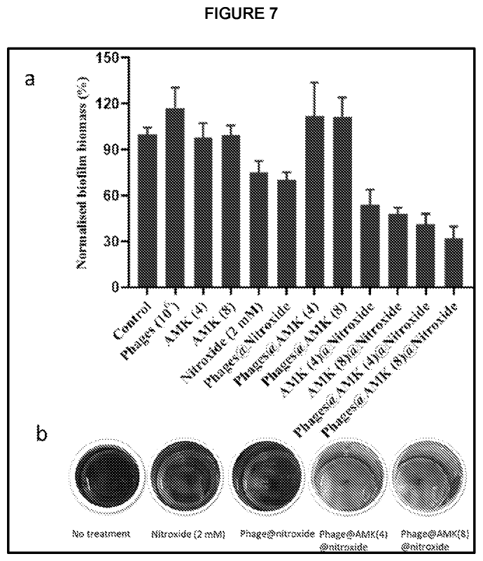

Graphs illustrating effects of antibiotic amikacin (AMK), nitroxide 4-amino-TEMPO, and phages on biofilm dispersal and biofilm inhibition are shown in FIG. 6. A graph illustrating the effects of different POEGA-b-PVBA conjugated formulations on biofilm dispersal are shown in FIG. 7.

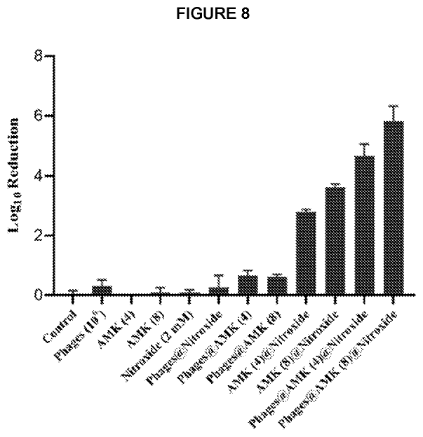

The cells in the treated and untreated groups were counted using the drop-plated method to determine the biofilm bacteria viability. Biofilms on the surface of the wells were rinsed with once 1 mL PBS and then were resuspended in 1 mL PBS. The 24-well plates were placed in an ultrasonication bath for 5 min before viable cells count assay. The re-suspended biofilm bacteria were serially diluted 10-fold (10⁻¹ to 10⁻¹⁰) in PBS, and 10 μL of each dilution was dropped onto MH agar plates in duplicate, which were incubated at 37° C. overnight. The results are presented as CFU mL⁻¹. A graph illustrating the effects of different POEGA-b-PVBA conjugated formulations on biofilm cell survival are shown in FIG. 8.

### Confocal Microscopy

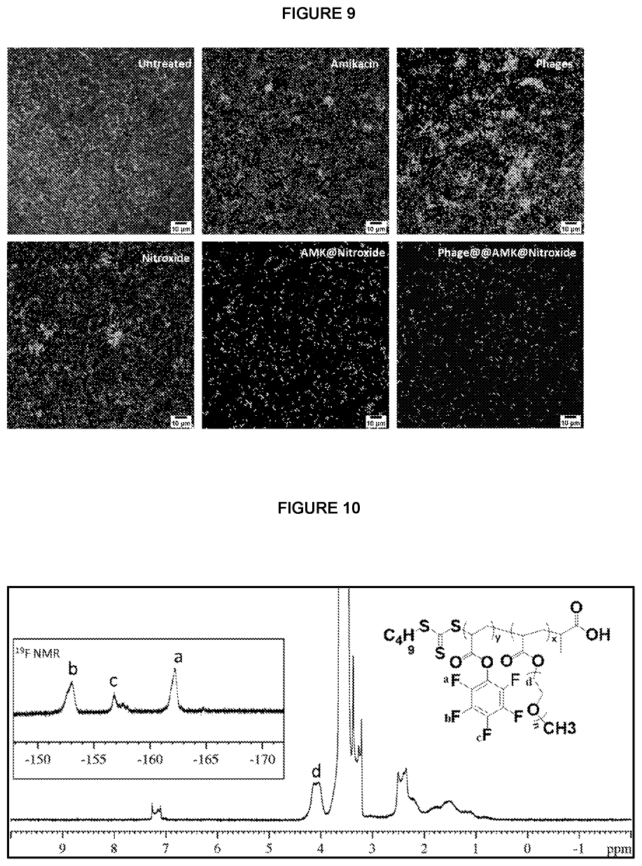

For microscopy analysis, P. aeruginosa biofilms were grown in glass-bottom 24-well plates (MatTek Corporation, Ashland MA, USA) for 6 h. After 3 h treatment, biofilms were rinsed twice with PBS before being stained with LIVE/DEAD® BacLight™ bacterial viability kit reagents (L-7007, Molecular Probes) according to the manufacturer's procedure. According to the manufacturer's procedure, 1 to 10 dilution of each of the two components was freshly prepared with PBS. 0.3 mL of the mixed solution was added to each well for 20 min incubation, and the plate was kept in the dark condition. The samples were analysed using confocal laser scanning microscopy (CLSM) with a Nikon A1 confocal microscope. Representative confocal images showing P. aeruginosa biofilms treated with POEGA-b-PVBA conjugated formulations are shown in FIG. 9.

### Data Analysis

GraphPad Prism 9 software was used for graphical representation and statistical analysis. Quantitative variables were compared using Student's t-test. A p-value of <0.05 was considered statistically significant. All values represent the average and standard deviation of two independent biological repeats. Data obtained in different treatment conditions were compared to the control samples taken at the same time point.

### (B) Comparative Example—Non-Cleavable Linker

The inventors conducted a comparative example to study the efficacy of phage-active agent conjugates wherein the active agent is covalently linked to the phage by a non-cleavable linker. The non-cleavable linkage is stable in acidic conditions such that the active agent will not be released at the infectious site. The non-cleavable linker comprises of a hydrophilic block (POEGA), which is closely related to polyethylene glycol to confer the hydrophilicity and biocompatibility properties, and a short poly (pentafluorophenyl acrylate) (PPFPA) block containing activated ester to react with amine-bearing active agents to form a non-cleavable amide bond.

### Synthesis of POEGA-b-PPFPA

Prepared POEGA macro-RAFT with 28 repeating units of OEGA per polymer molecule was chain extended with the pentafluorophenyl acrylate (PFPA) monomer. The POEGA macro-RAFT (0.5 g, 3.6×10⁻⁵ mol), PFPA (1.7×10⁻¹ g, 7.3×10⁻⁴ mol) and AIBN (1.2×10⁻³ g, 0.7×10⁻⁵ mol) were dissolved in toluene (3 ml) in a round bottom flask. The mixture was purged with nitrogen gas for 30 min to remove oxygen. Then the flask was placed in a pre-heated oil bath at 70° C. for 17 h. The reaction was terminated by placing the flask in an ice bath. The monomer conversion of the PFPA monomer and polymer composition were determined via ¹H NMR and ¹⁹F NMR using deuterated chloroform (CDCl₃) as the solvent (FIG. 10).

### Conjugation of Active Agents and Phage to POEGA-b-PPFPA

Stock solutions of copolymer POEGA-b-PPFPA (10 mg mL⁻¹), amikacin (AMK; 1 mg mL⁻¹) and 4-amino-TEMPO (nitroxide; 40 mM) were separately prepared by dissolving chemicals in Milli Q water and were stored at 4° C. for further use. Various treatment conditions were prepared and incubated at 37° C. with shaking at 180 rpm overnight before adding phages for further conjugation. The conjugation of phage on the POEGA-b-PPFPA was prepared with shaking at 180 rpm for 6 h before treatment application. The various treatment conditions are shown in Table 2.

TABLE 2
Preparation of POEGA-b-PPFPA phage conjugates

| | V_{polymer} | V_{AMK} | V_{nitroxide} | V_{phage suspension} | V media |
|---|---|---|---|---|---|
| Treatment (1 mL) | (ml) | (ml) | (ml) | (10⁹ PFU/ml) | (ml) |
| POEGA-b- | 0.4 | — | — | 0.02 | 0.58 |
| PPFPA@Phages | | | | | |
| POEGA-b- | 0.4 | 0.008 | — | — | 0.592 |
| PPFPA@AMK | | | | | |
| (8 μg/mL) | | | | | |
| POEGA-b- | 0.4 | — | 0.05 | — | 0.55 |
| PPFPA@Nitroxide | | | | | |
| POEGA-b- | 0.4 | 0.008 | — | 0.02 | 0.572 |
| PPFPA@Phages@AMK | | | | | |
| POEGA-b- | 0.4 | 0.008 | 0.05 | — | 0.542 |
| PPFPA@AMK | | | | | |
| (8 μg/mL)@Nitroxide | | | | | |
| POEGA-b- | 0.4 | 0.008 | 0.05 | 0.02 | 0.522 |
| PPFPA@Phages@AMK | | | | | |
| (8 μg/mL)@Nitroxide | | | | | |

### ¹H NMR Spectroscopy Analysis

The successful conjugation of phages and CIP nanoparticles were confirmed by ¹⁹F NMR using Varian 400-MR, Varian (400 MHZ) in D₂O (FIG. 12).

### Antimicrobial Assays

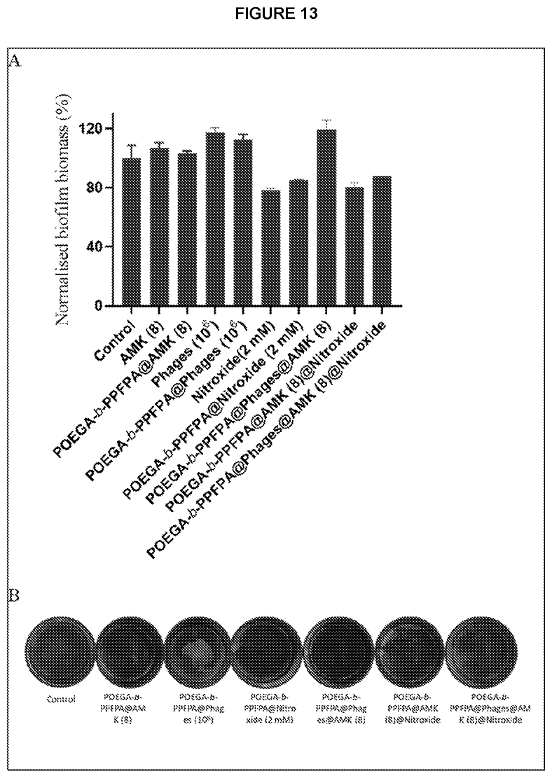

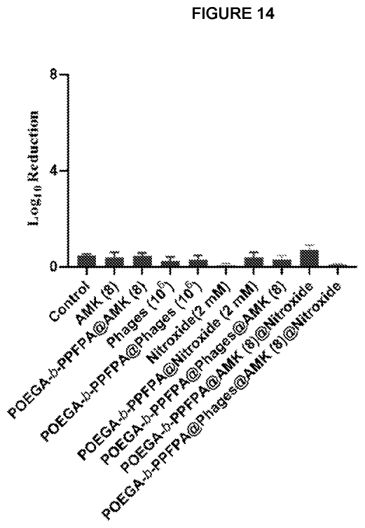

Biofilm dispersal and bacterial viability assays were conducted as described above using samples set out in Table 2. A graph illustrating the effects of different POEGA-b-PPFPA conjugated formulations on biofilm dispersal are shown in FIG. 13 and on biofilm cell survival are shown in FIG. 14.

### (C) Cleavable Linker—Non-Covalent Conjugation

The present invention also relates to phage-active agent conjugates wherein the active agent may have limited solubility in aqueous media and does not contain any functional groups to covalently bind to the linker. The active agent may be non-covalently encapsulated within a nanostructure comprising the linker. For example, one or more linkers may self-assemble into a nanostructure. The nanostructure thereby comprises one or more types of reactive functionality capable of forming a cleavable bond. The phage may be conjugated to the linker of the nanostructure via at least one of the one or more types of reactive functionality. Accordingly, the phage may be conjugated to the linker of the nanostructure and the active agent may be non-covalently linked to the linker. The nanostructure may be cleaved from the phage at the infectious site and the active agent may be released from the dynamic micellar nanostructures.

### Synthesis of Ciprofloxacin Nanoparticles and Phage-Ciprofloxacin Nanoparticle Conjugates

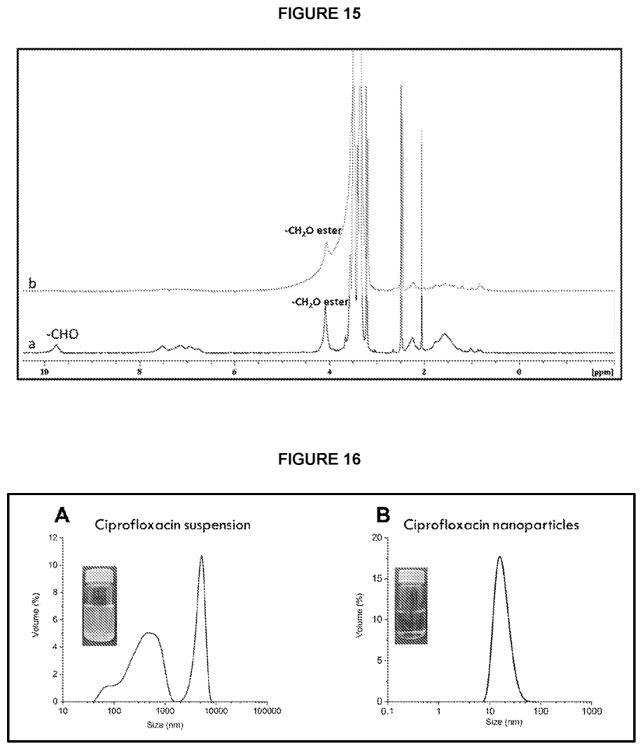

Stock solutions of diblock copolymer POEGA-b-PVBA (1 mL, 10 mg mL⁻¹) and ciprofloxacin (CIP) (1 mL, 1 mg mL⁻¹) were prepared separately by dissolving chemicals in Milli-Q water. Ciprofloxacin suspension was added dropwise into the POEGA-b-PVBA solution under moderate stirring overnight at room temperature. Water was then added slowly to the mixture to obtain final ciprofloxacin concentration of 0.1 mg mL⁻¹. Ciprofloxacin was encapsulated in the self-assembled micellar structure. The particle size of ciprofloxacin loaded nanoparticles (CIP-nanoparticles) will be determined by dynamic light scattering (DLS). CIP-nanoparticles with different CIP concentrations were prepared for further conjugation with phages via a cleavable Schiff base (imine) linkage by reacting primary amine groups on the phages with aldehyde groups of the diblock copolymer (the linker). The attachment of CIP-nanoparticles on phages was prepared with shaking at 180 rpm for 6 h before treatment. The titer of stock phage suspension is 10⁸ PFU mL⁻¹. Various treatment conditions if phage@CIP nanoparticle conjugates are shown in Table 3. The successful conjugation of CIP-nanoparticles with phages was determined by ¹H NMR (FIG. 15).

TABLE 3
Preparation of CIP nanoparticle phage conjugates

| | V_{CIP nanoparticles} (μl) | V_{phage suspension} (μl) | V media |
|---|---|---|---|
| Treatment (1 mL) | (1000 ng/mL) | (10⁶ PFU/ml) | (μl) |
| Phage only (10⁵ | 0 | 100 | 900 |
| PFU/ml) | | | |
| CIP (5 ng/ml) | 5 | 100 | 895 |
| nanoparticles- | | | |
| phage (10⁵ | | | |
| PFU/ml) | | | |
| CIP (10 ng/ml) | 10 | 100 | 890 |
| nanoparticle- | | | |
| phage (10⁵ | | | |
| PFU/ml) | | | |
| CIP (20 ng/ml) | 20 | 100 | 880 |
| nanoparticles - | | | |
| phage (10⁵ | | | |
| PFU/ml) | | | |
| CIP (40 ng/ml) | 40 | 100 | 860 |
| nanoparticles - | | | |
| phages (10⁵ | | | |
| PFU/ml) | | | |
| CIP (80 ng/ml) | 80 | 100 | 820 |
| nanoparticles- | | | |
| phage (10⁵ | | | |
| PFU/ml) | | | |

### ¹H NMR Spectroscopy Analysis

The successful conjugation of phages and CIP nanoparticles were confirmed by ¹H NMR using Varian 400-MR, Varian (400 MHz), in DMSO-de solvent (FIG. 15).

### Dynamic Light Scattering (DLS)

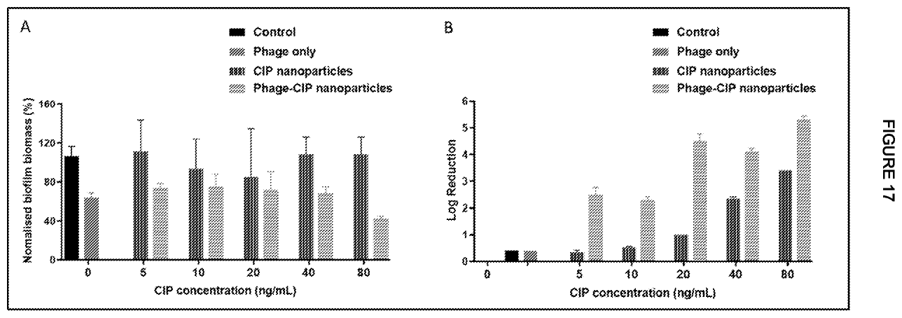

DLS was used to determine the particle size of self-assembled CIP nanoparticles. DLS measurements were performed using a Malvern Zetasizer Nano Series running DTS software and using a 4 mW He—Ne laser operating at a S4 wavelength of 633 nm and an avalanche photodiode (APD) detector. The scattered light was detected at an angle of 173º. The temperature was stabilized to +/−0.1° C. of the set temperature. To reduce the influence of larger aggregates the volume-average hydrodynamic particle size is reported. FIG. 16 shows dynamic light scattering (DLS) analysis of (a) CIP suspension and (b) self-assembled CIP nanoparticles (CIP concentration of 0.1 mg/mL in water).

### Antimicrobial Assays

The minimum inhibitory concentration (MIC) of the CIP, CIP nanoparticles, phage-CIP nanoparticle conjugates were measured using the broth microdilution technique. A bacterial culture medium with 10⁵-10⁶ CFU mL⁻¹ was prepared freshly in Mueller-Hinton (MH) broth and added to a 2-fold serial dilution of antimicrobial agents in 96-well microplates. A cell-only control and a medium-only control were also included in the same microplate. The 96-well microplates were incubated at 37° C. for 24 h. MIC was determined to be the concentration of antimicrobial agents at which the optical density (OD₆₀₀) was equal to that of a medium-only control using a multimode microplate reader (VICTOR™ X4, Perkin-Elmer). The MIC can also be recorded as the lowest concentration preventing visible bacterial growth after 24 h.

### Biofilm Formation Inhibition and Dispersal Assays

An E. coli inoculum was freshly prepared by inoculating one colony in 10 mL of LB broth and incubating on an orbital shaker at 220 rpm at 37° C. for 16-17 h. The bacterium culture medium was diluted 200-fold to an optical density at 600 nm (OD₆₀₀) between 0.003-0.005 in M9 minimal medium (containing 48 mM Na₂HPO₄, 22 mM KH₂PO₄, 9 mM NaCl, 19 mM NH₄Cl, 2 mM MgSO₄, 0.1 mM CaCl₂), and 20 mM glucose, pH 7.0). This medium was freshly prepared for each experiment.

For biofilm dispersal assay, the plates were incubated on an orbital shaker at 180 rpm for 6 h at 37° C. for biofilm formation. Planktonic bacteria were removed, and prepared samples (Table 3) and CIP nanoparticles were added in the well. The plates were incubated for further 4 h and 24 h before quantifying the biofilm biomass and viabilities of biofilm bacteria.

The biofilm biomass was analysed by crystal violet (CV) staining. The biofilm on each well surface of the plates was washed once with 1 mL PBS before CV staining. 1 mL of 0.1% CV solution made by a 1:20 dilution of 2% CV aqueous solution in PBS. The CV stained biofilm was washed twice with 1 mL PBS after 20 min incubation. The remaining CV-stained biofilm was dissolved in 33% glacial acetic acid and quantified by measuring the absorbance at 550 nm (OD₅₅₀) using a multimode microplate reader (VICTOR™ X4, Perkin-Elmer). All biofilm assays were performed in two independent experiments.

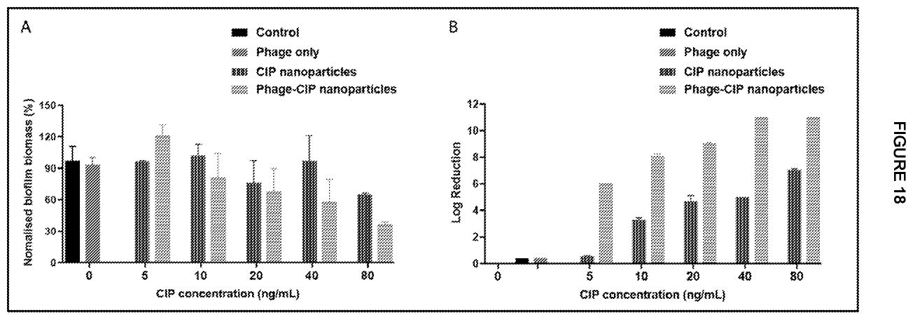

The cells in the treated and untreated groups were counted using the drop-plated method to determine the biofilm bacteria viability. Biofilms on the surface of the wells were rinsed with once 1 mL PBS and then were resuspended in 1 mL PBS. The 24-well plates were placed in an ultrasonication bath for 5 min before viable cells count assay. The re-suspended biofilm bacteria were serially diluted 10-fold (10-1 to 10-10) in PBS, and 10 μL of each dilution was dropped onto MH agar plates in duplicate, which were incubated at 37° C. overnight. The results are presented as CFU mL⁻¹. A graph illustrating the effects of different POEGA-b-PVBA non-covalently linked conjugates on biofilm dispersal are shown in FIG. 17 and on biofilm cell survival are shown in FIG. 18.

### Confocal Microscopy Analysis

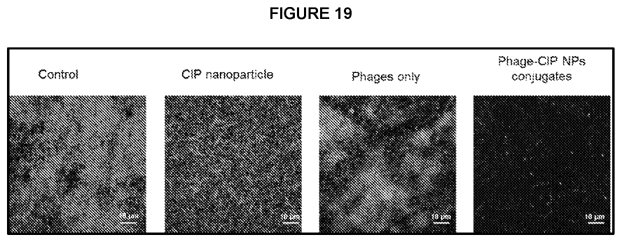

For microscopy analysis, E. coli biofilms were grown in glass-bottom 24-well plates (MatTek Corporation, Ashland MA, USA) for 6 h. After 3 h treatment, biofilms were rinsed twice with PBS before being stained with LIVE/DEAD® BacLight™ bacterial viability kit reagents (L-7007, Molecular Probes) according to the manufacturer's procedure. According to the manufacturer's procedure, 1 to 10 dilution of each of the two components was freshly prepared with PBS. 0.3 mL of the mixed solution was added to each well for 20 min incubation, and the plate was kept in the dark condition. The samples were analysed using confocal laser scanning microscopy (CLSM) with a Nikon A1 confocal microscope. The SYTO 9 and propidium iodide dyes presenting in green and red stains were shown viable cells and dead cells with damaged membranes, respectively. Representative confocal images showing E. coli biofilms treated with POEGA-b-PVBA non-covalently linked conjugates are shown in FIG. 19.

### Results and Discussion

### (A) Cleavable Linker-Covalent Conjugation

This study investigated engineering phages as nanobots for delivering therapeutic agents to infectious sites. To achieve a controlled release of therapeutic payloads, a diblock copolymer constituted of a hydrophilic block (POEGA), which is closely related to polyethylene glycol to confer the hydrophilicity and biocompatibility properties, and a short functional block containing aldehyde groups (PVBA) were used. Amikacin and the nitroxide 4-amino-TEMPO and phages were conjugated to the diblock copolymer via a cleavable Schiff base (imine) linkage by reacting primary amine groups of the amikacin, 4-amino-TEMPO and phage with aldehyde groups of the diblock copolymer. Formation of a cleavable imine conveniently allows for conjugation under mild conditions. Aldehyde groups can react rapidly with primary amines to yield a hydrolysable linkage that can allow slow release of the amikacin, 4-amino-TEMPO and/or phage in the mildly acidic microenvironment of biofilm. The imine is pH-labile, cleaved in mildly acidic conditions (pH typically ˜5.5), and thus may be suitable for the release of payloads in bacterial biofilms. AMK was selected in this study as a model drug due to its effective antipseudomonal activity against P. aeruginosa, this being both an important potential application and an ideal proof of principle. Drugs such as AMK are highly effective but sparingly used due to their toxicity.

### Synthesis of POEGA-b-PVBA Diblock Copolymer as a Functional Linker

POEGA-b-PVBA was synthesised using cleavable addition fragmentation chain transfer (RAFT) polymerisation according to Scheme 1. The first hydrophilic block, poly(oligo(ethylene glycol) methyl ether acrylate) (POEGA) macro-RAFT agent, was prepared in toluene at 70° C. in the presence of 2-(butylthiocarbonothioylthio) propionic acid (PABTC) as a RAFT agent. OEGA monomer conversion was calculated using ¹H NMR analysis by comparing the signals of the vinyl proton peaks (at 6.3 ppm) to that of ester —OCH₂ proton peaks (at 4.2 ppm) (FIG. 1(a)). After 17 h polymerisation, approximately 83% monomer conversion, equivalent to 28 repeating units of OEGA per polymer molecule, was achieved. The molecular weight of POEGA macro-RAFT obtained from GPC was in good agreement with the theoretical molecular weight (M_{n,theo}=11900 g mol-1, and M_{n,GPC}=9000 g mol-1 with a low polydispersity index, PDI=1.19). The chain extension of POEGA was successful according to the ¹H NMR and GPC analysis. ¹H NMR analysis of purified POEGA-b-PVBA polymer (FIG. 1(c)) confirmed the presence of VBA by the appearance of the characteristic signal at 9.8 ppm attributed to the aldehyde proton. The percentage of VBA monomer conversion was around 70% by comparison of the signal of the aldehyde proton peak of VBA monomer at 9.8 ppm or the vinyl signals at 5-6 ppm to that of ester —OCH₂ proton peak before and after VBA polymerisation. A shift to higher molecular weight from GPC analysis (M_{n,GPC}=15 000 g mol⁻¹, with a low polydispersity index, PDI=1.19) further confirmed the successful PVBA incorporation into the copolymer (FIG. 4). The number of repeating units of VBA was 10 based on ¹H NMR analysis. The final diblock copolymer comprised of OEGA (28 repeating units) to confer the hydrophilicity and biocompatibility and a short aldehyde block (10 units) to ensure the water solubility of the polymer.

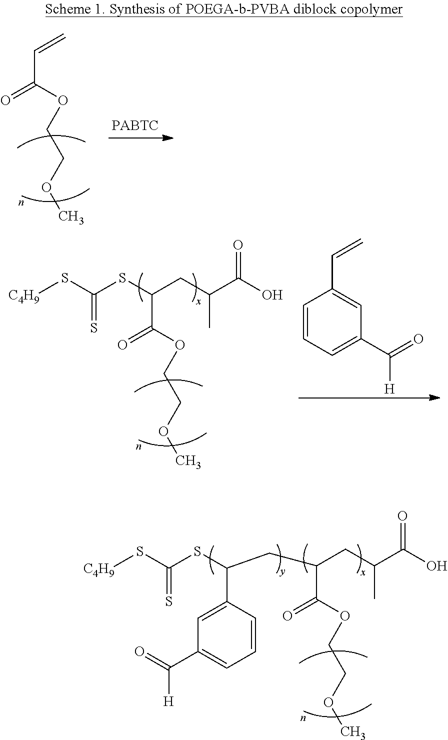

Conjugation of Antibiotic and Nitroxide onto Phages Using the Diblock Copolymer as a Cleavable Linker

AMK and 4-amino-TEMPO were conjugated onto the polymer via Schiff base/imine of amine groups of the drug and aldehyde groups of the POEGA-b-PVBA. Unreacted aldehyde groups were employed to attach the phage capsid to the antibiotics-conjugated polymer. The negative charge of the phage head may advantageously facilitate the attachment of positively charged polymer-antibiotic conjugate on the phage head and orient tail fibres outwards to facilitate host recognition.

Determining the individual conjugation efficiency with the linker in the presence of antibiotics, nitroxide and phages is complex. Successful attachment of bacteriophages, nitroxide and antibiotics on the copolymer linker was individually confirmed via ¹H NMR analysis by monitoring the decrease in the signal intensity at 9.8 ppm, which corresponds to the aldehyde group. The conjugation efficiency of the nitroxides and the linker was 85%, the phages and the linker was 96% and the AMK and the linker was 100%, as determined by comparing the intensity of aldehyde signal at 9.8 ppm with the unchanged —CH₂O ester of OEGA at 4.2 ppm before and after the reaction (FIGS. 2(b)-2(d)). Conjugation was also confirmed by ATR-FTIR spectroscopy. After the conjugation with phages, signals from the aldehyde bond at 1728 cm⁻¹ disappeared and new absorption peaks appeared at 1643 cm⁻¹, corresponding to the characteristic signal of the imine bond. The same approach was used to confirm the conjugation of antibiotics and nitroxides onto the linker (FIG. 5). In this study, the amount of linker was calculated so that the slight excess of the linker was achieved to ensure the high conjugation efficiency of therapeutic agents.

Successful release of phages, AMK and nitroxide from respective POEGA-b-PVBA conjugates in acidic media (pH 5.5) was individually confirmed via ¹H NMR analysis by monitoring the increase in the signal intensity at 9.8 ppm, which corresponds to the POEGA-b-PVBA aldehyde group (FIGS. 3(a)-3(c)).

### Effects on Biofilm Dispersal and Bacteria Viability of Antibiotic, Nitroxide and Phages Alone

First, the effects of AMK, 4-amino-TEMPO and phages alone on biofilm dispersal and viability of established bacterial colonies was investigated. P. aeruginosa biofilms were grown in vitro for 6 h before being separately exposed to AMK (4-32 μg mL⁻¹), nitroxide (0.25-2 mM) and phages (10²-10⁸ PFU/ml) for 2 h (FIG. 4A).

The results are shown in FIG. 6(a) (biofilm dispersal) and FIG. 6(b) (biofilm inhibition). AMK inhibited biofilm formation at a concentration of 4 μg mL⁻¹, but could only induce a 10% reduction of biofilm biomass of pre-established P. aeruginosa biofilm at a high concentration (32 μg mL⁻¹, 8×MIC). AMK at a higher concentration than 4 μg mL⁻¹ showed a strong effect on inhibiting the formation of biofilms. Treatment with phages alone (in a range of 10⁶-10⁸ PFU mL 1) did not show significant dispersal for the pre-established P. aeruginosa biofilm. However, phages at a titer higher than 10⁴ PFU mL⁻¹ began to exhibit an inhibitory effect on bacterial biofilm growth. The phages may play an important role in the control of biofilm formation by P. aeruginosa. Treatment with nitroxide agent alone at higher concentrations of 2 and 5 mM induced 30% and 70% P. aeruginosa biofilm dispersal, respectively. Nitroxide treatment (concentration range of 1 to 5 mM) also significantly inhibited biofilm formation. The MIC value for 4-amino-TEMPO was found to be 6 mM. These data show that AMK, 4-amino-TEMPO and phages alone can inhibit bacterial biofilm formation, but had a weak effect on the dispersal of pre-established biofilm.

### Effects of Cotreatment on Biofilm Dispersal and Biofilm Cell Survival

The co-treatment of phages and antibiotics has been increasingly studied in in vitro grown bacterial biofilm. The combination of phages and antibiotics may result in additive, synergistic, antagonistic or no effect. Few studies have reported synergy between phage and antibiotic activities. However, antagonistic effects could arise, for example due to antibiotic interference with phage infection or the preferential phage infection on antibiotic-sensitive bacteria compared to antibiotic-resistant biofilm. In the case of the phage-drug conjugates described herein, it is important to note that even if the combination of phages and one or more types of active agent in the conjugate form has a similar effect to the phages and active agents separately, an advantage of using phage-drug conjugates is to utilise the phages as nanobots to deliver the active agents to targeted sites while avoiding systemic toxicity.

To investigate the potential synergistic effects of cotreatment with AMK, 4-amino-TEMPO and/or phages, the following groups were prepared to assess biofilm dispersal and biofilm viability of Pseudomonas aeruginosa: 1. Control bacteria, 2. AMK alone, 3. Nitroxide (4-amino-TEMPO) alone, 4. Phages alone, 5. Phage-AMK conjugates, 6. Phage-nitroxide conjugates, 7. AMK-nitroxide conjugates, 8. Phage-AMK-nitroxide conjugates, and 9. PBS only. From the data in FIG. 6, AMK concentrations of 4 and 8 μg ml⁻¹ (at 1× and 2×MIC), nitroxide concentration of 2 mM, and the phage titer of 10⁶ PFU mL⁻¹ were chosen for this study. The effect of the combined treatments in which antibiotic concentrations were equal to and greater than MIC for targeted bacteria were explored.

The results of the biofilm dispersal assay are shown in FIG. 7(a), with photographs of CV-stained biofilms without treatment and after treatment are shown in FIG. 7(b). Treatment of P. aeruginosa biofilms with AMK (4 or 8 μg/ml) or phage alone did not have an effect on biofilm dispersal. The low level of biofilm dispersal exhibited by the phages alone may be due to their limited penetration, restricting accessibility to the receptors. Treatment with phage-nitroxide or AMK-nitroxide conjugates was about as effective as the use of nitroxide alone in the reduction of biofilm biomass (about 30%), whereas the phage-AMK-nitroxide conjugate exhibited a greater reduction in biofilm biomass (about 70%).

The results of the biofilm cell survival assay are shown in FIG. 8. Treatment with a single component (AMK, nitroxide or phage alone) did not affect the survival of biofilm bacteria. While nitroxide alone was shown to induce biofilm dispersal, it had an insignificant effect on biofilm bacteria. Treatment with phage-AMK or phage-nitroxide conjugates was more effective than single agent alone, exhibiting a 40-60% decrease in biofilm bacteria. Finally, treatment with phage-AMK-nitroxide conjugates was even more effective, reducing bacteria cell numbers in the biofilm by more than 5 log units relative to untreated control. The higher the concentration of AMK, the more significant reduction in biofilm bacteria cells was observed. These results suggest that the ability of nitroxide to disperse the biofilm may result in a synergistic effect between phages and antibiotics in bactericidal activity, which is promising for combatting biofilm-related infections.

Confocal microscopy was used to visualize biofilm dispersal and bacterial viability. P. aeruginosa biofilms were stained with LIVE/DEAD dyes, where viable and non-viable cells appear green and red respectively. While cultures treated with nitroxide at 2 mM displayed greatly reduced biofilm biovolume, cultures treated with nitroxide and amikacin conjugates and phages, amikacin and nitroxide conjugates exhibited more dead cells, compared with untreated control biofilms or those inoculated with the phase and amikacin alone (FIG. 9). Overall, the crystal violet and confocal microscopy results confirmed that phages, amikacin and nitroxide can effectively disperse biofilms of P. aeruginosa and reduce the viability of P. aeruginosa to a comparable or greater extent than any individual components of the conjugate alone.

### (B) Comparative Example—Non-Cleavable Linker

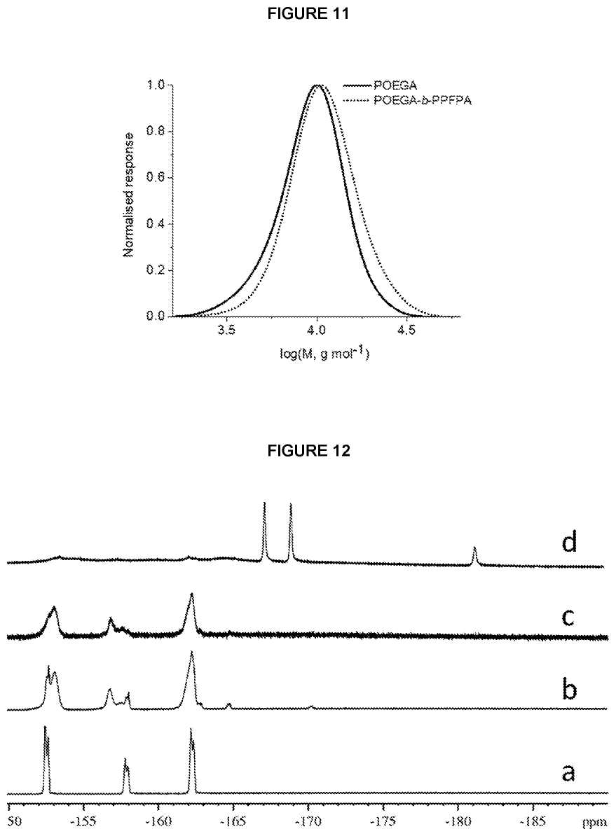

The chain extension of POEGA (M_{n,SEC}=9000 g mol⁻¹ with a low polydispersity index, PDI=1.19) with PFPA monomer was successful according to ¹H NMR and ¹⁹F NMR analysis. The percentage of PFPA monomer conversion determined from ¹H NMR was about 70% by comparing the signal of the vinyl signals at 5-6 ppm to that of ester —OCH₂ proton peak before and after polymerization. The number of repeating units of PFPA was 14 based on ¹H NMR analysis. The final diblock copolymer comprised of POEGA (28 repeating units) to confer the hydrophilicity and biocompatibility and a short aldehyde block (10 units) to ensure the water solubility of the polymer. The final diblock copolymer comprised of POEGA (28 repeating monomer units) to confer the hydrophilicity and biocompatibility and a shorter-activated ester PPFPA block (14 repeating monomer units) to ensure the water solubility of the polymer. The presence of ¹H and ¹⁹F NMR signals attribute to both POEGA and PPFPA confirmed the successful chain extension. ¹⁹F NMR analysis (FIG. 10) confirmed the expected signals at -153, -157, and -162 ppm attributable to the PPFPA (inset). A shift to higher molecular weight from SEC analysis (M_{n,GPC}=10 300 g mol⁻¹, with a low polydispersity index, PDI=1.21) further confirmed the successful PPFPA incorporation into the copolymer (FIG. 11).

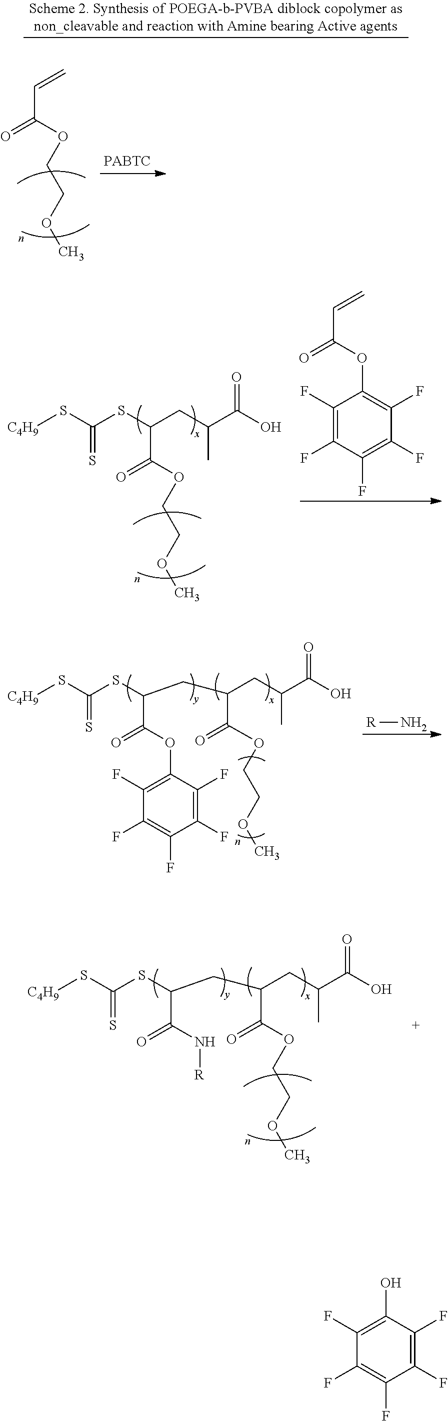

The attachment of agents was determined by ¹⁹F NMR (FIG. 12). ¹⁹F NMR spectra of the purified POEGA-b-b-PPFPA linker, POEGA-b-b-PPFPA conjugated with phages are shown in FIG. 12. ¹⁹F NMR spectroscopy showed the disappearance of signals at -153, -157, and -162 ppm attributable to the PPFPA and the appearance of new signals at -165, -172, and -178 ppm from the released pentafluorophenol, confirming the successful conjugation with 100% conjugation efficiency.

The results are shown in FIG. 13 (biofilm dispersal) and FIG. 14 (biofilm cell viability). As can be seen from FIG. 13A, treatment with nitroxide agent alone at AMK concentration of 8 μg/mL induced about 30% P. aeruginosa biofilm dispersal. All other samples did not show significant biofilm dispersal. The results of the biofilm dispersal assay are shown in FIG. 13B, with photographs of CV-stained biofilms without treatment and after treatment. Treatment of P. aeruginosa biofilms with conjugates using non-cleavable linker did not have an effect on biofilm dispersal. Similarly, treatment with all the formulations did not affect the survival of biofilm bacteria. This comparative data demonstrates an advantage of the cleavable linker over the non-cleavable linker in biofilm dispersal and biofilm cell viability.

### (C) Cleavable Linker-Non-Covalent Conjugation

Successful attachment of CIP nanoparticles onto phages was confirmed from ¹H NMR analysis by monitoring the decrease in the signal intensity at 9.8 ppm, which corresponds to the aldehyde group. The conjugation efficiency was 100%, as determined by comparing the intensity of aldehyde signal at 9.8 ppm with the unchanged-CH₂O ester of POEGA block at 4.2 ppm before (FIG. 15a) and after the reaction (FIG. 15b).

Ciprofloxacin shows a poor solubility in water with large aggregates to form a cloudy suspension (FIG. 16A). Thus, CIP was encapsulated in nanostructures to improve its solubility. In this experiment, water was added slowly to the stirred POEGA-b-PVBA polymer and CIP suspension to yield micelles. The self-assembly of amphiphilic diblock copolymers in water was confirmed via DLS. The mean particle size of CIP nanoparticles in water, as derived from DLS data, was ca. 20 nm (by volume-average).

While the MIC values of CIP nanoparticles were found to be 10 μg mL⁻¹, the poor solubility of CIP in water influences the MIC measurements with varied MIC values (Table 4). Thus, CIP suspension was not used for further E. coli biofilm dispersal and bacteria viability assays.

TABLE 4
Minimum inhibitory concentration (MIC,
ng/mL) of CIP and CIP nanoparticles

| Repeat | Ciprofloxacin | Ciprofloxacin - nanoparticles |
|---|---|---|
| 1 | 20 | 10 |
| 2 | 40 | 10 |
| 3 | 20 | 10 |

### Effects on Biofilm Dispersal and Bacteria Viability of CIP Nanoparticles, Phages Alone and Phage-CIP Nanoparticle Conjugates

First, the effects of CIP nanoparticles and phages alone on biofilm dispersal and viability of established bacterial biofilm was investigated. E. coli biofilms were grown in vitro for 6 h before being separately exposed to CIP nanoparticles (CIP concentration of 5, 10, 20, 40, 80 ng mL⁻¹) and E. coli phages (10⁵ PFU/mL) for 4 h (FIG. 17) or 24 h (FIG. 18).

The results are shown in FIG. 17A (biofilm dispersal) and FIG. 17B (biofilm cell viability). Treatment with phages alone (10⁵ PFU mL⁻¹) could induce a 60% reduction of biofilm biomass of pre-established E. coli biofilm after 4 h incubation. However, CIP nanoparticles even at a high CIP concentration (80 ng mL⁻¹, 8×MIC) did not show significant biofilm dispersal. Treatment with phage-CIP nanoparticle conjugates exhibited about 70% reduction in biofilm biomass at CIP concentration of 80 ng mL⁻¹, 8×MIC. As can be seen in FIG. 18A, treatment with CIP nanoparticles alone could induce a 40% reduction of biofilm biomass of pre-established E. coli biofilm after 24 h incubation at high CIP concentration (80 ng mL⁻¹, 8×MIC). However, after 24 h treatment with phages alone, increased biofilm biomass and thicker biofilm was observed, possibly due to the development of phage resistance. Interestingly, longer incubation time did not affect the biofilm dispersal ability of phage-CIP nanoparticle conjugates, about 70% reduction in biofilm biomass was observed at CIP concentration of 80 ng mL⁻¹, 8×MIC.

The results of the biofilm cell survival assay are shown in FIGS. 17B and 18B. As can be seen in FIG. 17B, treatment with phages alone for 4 h did not affect significantly on the survival of biofilm bacteria. While CIP nanoparticles alone did not induce significant biofilm dispersal, it had a significant effect on biofilm bacteria viability at high CIP concentrations. The higher the concentration of CIP, the more log reduction in biofilm bacteria cells was achieved. Treatment with highest CIP concentration (80 ng mL⁻¹, 8×MIC) reduced bacteria cell numbers in the biofilm by 3 log units relative to untreated control. Treatment with phage-CIP nanoparticle conjugates was more effective in killing biofilm bacteria with more than 4 log reduction at CIP concentration of 20 ng mL⁻¹ (2×MIC) and about 5 log reduction at CIP concentration of 80 ng mL⁻¹ (8×MIC). Longer incubation time (24 h) of both CIP nanoparticles and phage-CIP nanoparticle conjugates significantly affect E. coli biofilm bacterial viability (FIG. 18B). The higher CIP concentration resulted in greater significant reduction in biofilm bacterial cells, consistent with the clinically important concentration-dependent killing characteristics. Treatment with CIP nanoparticles and phage-CIP nanoparticle conjugates was more effective in killing biofilm bacteria with more than 6 and 9 log reduction respectively at CIP concentration of 80 ng mL⁻¹ (8×MIC). These results suggest that the combination of phages and CIP nanoparticles with improved solubility in aqueous media may result in a synergistic effect between phages and CIP in biofilm dispersal and bactericidal activity, which is effective for biofilm-related infections.

Confocal microscopy was used to visualize biofilm dispersal and bacterial viability. E. coli biofilms were stained with LIVE/DEAD dyes and viable and non-viable cells appear green and red respectively. While cultures treated with CIP nanoparticles (CIP concentration of 20 ng/ml, 2×MIC) for 4 h did now show significant biofilm dispersal which is in a good agreement with the data obtained from crystal violet assay (FIG. 17A). More dead cells were observed when the cultures were treated with phage alone. Phage-CIP nanoparticle conjugates displayed greatly reduced biofilm biovolume, cultures treated with conjugates exhibited more dead cells, compared with untreated control biofilms or those treated with the phages and CIP nanoparticles alone (FIG. 19). Overall, the crystal violet and confocal microscopy results confirmed that phage-CIP nanoparticle conjugates can effectively disperse E. coli biofilms and reduce the bacterial viability to greater extent than individual components.

1. A conjugate of a phage linked with one or more types of active agent by a linker, wherein the phage is conjugated to the linker by one or more types of cleavable bond and the one or more types of active agent are each bound to the linker.

2. The conjugate of claim 1, wherein the phage and the one or more types of active agent are each independently conjugated to the linker by one or more types of cleavable bond.

3. The conjugate of claim 1, wherein the one or more types of active agent are each indirectly bound to the linker.

4. The conjugate of claim 3, wherein the one or more types of active agent are each encapsulated within a nanostructure comprising the linker, thereby the one or more types of active agent are each indirectly bound to the linker.

5. The conjugate of any one of claims 1 to 4, wherein at least one of the one or more types of cleavable bond is pH labile.

6. The conjugate of any one of claims 1 to 5, wherein at least one of the one or more types of cleavable bond is selected from a hydrazone, an imine, an acetal, a ketal, and an ester.

7. The conjugate of any one of claims 1 to 6, wherein the linker comprises a polymer which comprises the one or more types of cleavable bond.

8. The conjugate of claim 7, wherein the polymer comprises a diblock copolymer comprising a first block and a second block.

9. The conjugate of claim 8, wherein the first block comprises poly(oligo(ethylene glycol) methyl ether acrylate) (POEGA).

10. The conjugate of claim 7 or claim 8, wherein the second block comprises a polymer composed of a repeating monomer unit having the following structure: 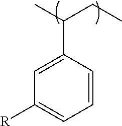
wherein
R is independently —CHO or —C═NZ; and
Z is independently a phage or an active agent.

11. The conjugate of any one of claims 1 to 10, wherein the conjugate has the following structure: 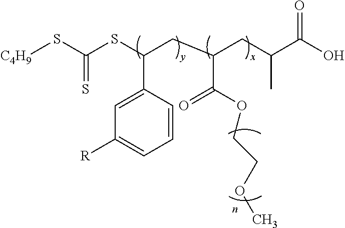
wherein R is independently —CHO or —C═NZ;
Z is independently a phage or an active agent;
n is 9;
x is 10 to 30; and
y is 5 to 15.

12. The conjugate of any one of claims 1 to 11, wherein the phage is capable of infecting one or more pathogens selected from Escherichia coli, Enterococcus faecium, Staphylococcus aureus, Klebsiella pneumoniae, Acinetobacter baumannii, Pseudomonas aeruginosa and Enterobacter spp.

13. The conjugate of any one of claims 1 to 12, wherein the phage is selected from a filamentous phage, an MS2 phage and a caudovirus.

14. The conjugate of claim 13, wherein the phage is a caudovirus.

15. The conjugate of any one of claims 1 to 14, wherein the one or more types of active agent comprise one or both of (i) one or more antibacterial agents and (ii) one or more antibiofilm agents.

16. The conjugate of claim 15, wherein the one or more antibacterial agents comprise one or more of the following: an aminoglycoside antibiotic, an antimicrobial peptide and a fluoroquinolone antibiotic.

17. The conjugate of claim 15 or claim 16, wherein the one or more antibiofilm agents comprise one or both of the following: a nitric oxide (NO) donor and a nitroxide-containing antibiofilm agent.

18. A method for preparing a conjugate of a phage linked with one or more types of active agent by a linker, the method comprising:
providing a linker comprising one or more types of reactive functionality capable of forming a cleavable bond;
binding one or more types of active agent to the linker; and
conjugating a phage to the linker via at least one of the one or more types of reactive functionality;
thereby providing the conjugate, wherein the phage is conjugated to the linker by one or more types of cleavable bond and the one or more types of active agent are each bound to the linker.

19. The method of claim 18, comprising:
conjugating one or more types of active agent to the linker, each active agent independently being conjugated to the linker via at least one of the one or more types of reactive functionality;
thereby providing the conjugate, wherein the phage and the one or more types of active agent are each independently conjugated to the linker by one or more types of cleavable bond.

20. The method of claim 18, comprising:
encapsulating one or more types of active agent within a nanostructure comprising the linker, each active agent independently being indirectly bound to the linker;
thereby providing the conjugate, wherein the phage is conjugated to the linker by one or more types of cleavable bond and the one or more types of active agent are each indirectly bound to the linker.

21. The method of any one of claims 18 to 20, wherein at least one of the one or more types of reactive functionality is capable of forming a cleavable bond that is pH labile.

22. The method of any one of claims 18 to 21, wherein at least one of the one or more types of reactive functionality is selected from an aldehyde, a ketone, a hydrazine, an amine, an alcohol, and a carboxylic acid.

23. The method of any one of claims 18 to 22, wherein the linker comprises a polymer which comprises the one or more types of reactive functionality.

24. The method of claim 23, wherein the polymer comprises a diblock copolymer comprising a first block and a second block.

25. The method of claim 24, wherein the first block comprises poly(oligo(ethylene glycol) methyl ether acrylate) (POEGA).

26. The method of claim 24 or claim 25, wherein the second block comprises poly(3-vinylbenzaldehyde) (PVBA).

27. The method of any one of claims 18 to 26, wherein the linker has the following structure: 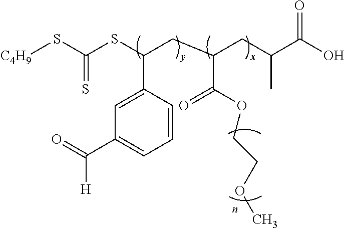
wherein
n is 9;
x is 10 to 30; and
y is 5 to 15.

28. A conjugate prepared by the method of any one of claims 18 to 27.

29. A pharmaceutical composition comprising the conjugate of any one of claims 1 to 17 or prepared by the method of any one of claims 18 to 27, and a pharmaceutically acceptable diluent, excipient or carrier.

30. A method for preventing or treating a bacterial infection, the method comprising administering the conjugate of any one of claims 1 to 17, the conjugate prepared by the method of any one of claims 18 to 27, or the pharmaceutical composition of claim 29 to an individual in need thereof, thereby preventing or treating the bacterial infection.

31. The method of claim 30, wherein the individual is susceptible to developing a bacterial infection.

32. The method of claim 31, wherein the individual has cystic fibrosis.

33. The method of claim 31, wherein the individual has a prosthetic medical device.

34. A method for preventing or treating a cancer, the method comprising administering the conjugate of any one of claims 1 to 17, the conjugate prepared by the method of any one of claims 18 to 27, or the pharmaceutical composition of claim 29 to an individual in need thereof, thereby preventing or treating the cancer.
